(19) United States
(12) Patent Application Publication
(10) Pub. No.: US 2018/0200206 A1
(43) Pub. Date: Jul. 19, 2018

(54) TRANSPAPILLARY METHODS AND COMPOSITIONS FOR TREATING BREAST DISORDERS

(71) Applicant: ATOSSA GENETICS INC., Seattle, WA (US)

(72) Inventor: Steven C. Quay, Seattle, WA (US)

(73) Assignee: Atossa Genetics Inc., Seattle, WA (US)

(21) Appl. No.: 15/744,004

(22) PCT Filed: Jul. 14, 2016
(86) PCT No.: PCT/US2016/042202

§ 371 Date: Jan. 11, 2018

Related U.S. Application Data
(60) Provisional application No. 62/192,505, filed on Jul. 14, 2015.

(51) Int. Cl. A61K 31/138; A61K 9/00; A61K 31/202; A61K 31/592; A61K 31/593; A61K 45/06; A61P 35/00

(52) U.S. Cl. CPC A61K 31/138; A61K 9/0041; A61K 31/202; A61K 2300/00; A61K 31/593; A61K 45/06; A61P 35/00; A61K 31/592

(57) ABSTRACT

Methods and treatments are taught for the treatment of breast disorders, including proliferative breast disease, breast cancer, and increased breast density. The methods and compositions deliver efficacious formulations of chemical and/or biological treatment medicaments to the breast via a transpapillary route.

### CROSS-REFERENCE

This application claims priority to U.S. Provisional Patent Application No. 62/192,505, filed Jul. 14, 2015, the entirety of which is hereby incorporated by reference.

### BACKGROUND

Breast cancer is by far the most common form of cancer in women, and it is the second leading cause of cancer death in humans. Despite advances in diagnosing and treating breast cancer, the prevalence of this disease has been steadily rising at a rate of about 1% per year since 1940. Today, the likelihood that a women living in North America will develop breast cancer during her lifetime is one in eight.

Breast disorders include breast cancers and benign but often precancerous lesions, such as ductal hyperplasia, lobular hyperplasia, atypical ductal hyperplasia, and atypical lobular hyperplasia. Breast cancers include any malignant tumor of breast cells. There are several types of breast cancer. Exemplary breast cancers include, but are not limited to, ductal carcinoma in situ, lobular carcinoma in situ, invasive (or infiltrating) ductal carcinoma, invasive (or infiltrating) lobular carcinoma, inflammatory breast cancer, triple-negative breast cancer, ER+ breast cancer, HER2+ breast cancer, adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, and micropapillary carcinoma. A single breast tumor can be a combination of these types or be a mixture of invasive and in situ cancer.

Current best practice for the treatment of breast cancer is to diagnose breast cancer with mammography and then treat the patient with surgery, radiation therapy, and chemotherapy. The current widespread use of mammography has resulted in improved detection of breast cancer. Nonetheless, the death rate due to breast cancer has remained unchanged at about 27 deaths per 100,000 women. All too often, breast cancer is discovered at a stage that is too far advanced, when therapeutic options and survival rates are severely limited.

Adjuvant therapy via oral delivery, for example with tamoxifen, is known to have severe side effects. Although an exciting new method of delivering drugs to treat breast conditions is transpapillary method of delivery via the mammary papillae (U.S. Pat. Pub. No. 20140088059; U.S. Pat. App. No. 61/926,180; PCT/US15/10808), there remains a need for improved methods for treating breast disorders such as hyperplasia and breast cancer.

### SUMMARY OF THE INVENTION

Described herein, in certain embodiments, are methods of delivering a composition comprising endoxifen or a pharmaceutically acceptable salt thereof to a breast duct of an individual in need thereof, comprising: contacting the composition contained within a treatment chamber of a device with a nipple of a breast; and applying positive pressure on the composition.

In some embodiments, the composition is forced into 1 to 5 breast ducts. In some embodiments, the composition is forced into 4 to 8 breast ducts. In some embodiments, the composition is forced into 7 to 11 breast ducts. In some embodiments, the individual has a breast disorder. In some embodiments, the breast disorder is proliferative breast disease, breast cancer, or increased breast density. In some embodiments, the proliferative breast disease is atypical ductal hyperplasia or atypical lobular hyperplasia. In some embodiments, the breast is cancer ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is ER+ breast cancer, HER2+ breast cancer, or triple-negative breast cancer. In some embodiments, the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma.

In some embodiments, the individual is tamoxifen resistant. In some embodiments, the individual has been predicted to have a (i) moderate to high risk of cancer relapse or (ii) low to moderate rate of disease-free survival. In some embodiments, the moderate or high risk of cancer relapse is due to reduced expression or reduced function of a member of cytochrome P450 family. In some embodiments, the member of cytochrome P450 family is CYP2D6, CYP2B6, CYP2C9, CYP2C19, CYP3A4, or CYP3A5. In some embodiments, the individual has immune suppression. In some embodiments, the individual has a high risk of tumor escape. In some embodiments, the individual has increased activity or expression of IDO1, IDO2, TDO, or a combination thereof. In some embodiments, the endoxifen is an E-isomer, a Z-isomer or a mixture thereof.

In some embodiments, the composition comprises endoxifen or a pharmaceutically acceptable salt thereof at a concentration ranging from 0.01% to 15% by weight of the composition. In some embodiments, the composition comprising endoxifen or a pharmaceutically acceptable salt thereof is formulated in a hydroalcoholic gel, a hydroalcoholic solution, a patch, a cream, an emulsion, a lotion, an ointment, a powder, a paste, or an oil. In some embodiments, the composition further comprises at least one therapeutic agent. In some embodiments, the composition further comprises a plurality of therapeutic agents. In some embodiments, the therapeutic agent is selected from the group consisting of alkylating agents, anti-neoplastics, anti-mimetics, anti-metabolites, antitumor antibiotics, topoisomerase inhibitors, mitotic inhibitors, corticosteroids, differentiating agent, anti-cancer antibodies, immunotherapy agents, anthracyclins, platinums, vinca alkoids, camptothecins, taxanes, hormones, 1-alpha-hydroxylase inhibitors, 24-hydroxylase inhibitors, or a combination thereof. In some embodiments, the composition further comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, 4-hydroxytamoxifen, N-desmethyltamoxifen, endocoxifen, lasofoxifene, raloxifene, benzothiophene, bazedofoxifene, arzoxifene, miproxifene, levormeloxifene, droloxifene, clomifene, idoxifene, EM652, ERA-92, toremifene, trastuzumab, vinorelbine, butyric acid, epirubicin, doxorubicin, and combinations thereof. In some embodiments, the therapeutic agent is a tryptophan metabolism inhibitor, a kynurenine depletor, an inhibitor of IDO1, IDO2, TDO, or a combination thereof. In some embodiments, the therapeutic agent is an inhibitor of IDO1, IDO2, TDO, or a combination thereof. In some embodiments, the therapeutic agent is a PD-L1 inhibitor, a PD-1 inhibitor, a CTLA-4 inhibitor, or a combination thereof. In some embodiments, the composition further comprises at least one omega-3 fatty acid, at least one vitamin D compound or a pharmaceutically acceptable salt thereof, or a combination thereof.

In some embodiments, the composition comprises (i) endoxifen or a pharmaceutically acceptable salt thereof; (ii) at least one an omega-3 fatty acid; and (iii) at least one vitamin D compound or a pharmaceutically acceptable salt thereof. In some embodiments, the composition comprises the omega-3 fatty acid at a concentration ranging from 10% to 90% by weight of the composition. In some embodiments, the omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof. In some embodiments, the omega-3 fatty acid is a triglyceride or a phospholipid. In some embodiments, the composition further comprises a mixture of EPA and DHA. In some embodiments, the vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4, 1,25 dihydroxy-19-nor-vitamin D2, 1-alphahydroxyvitamin D3, vitamin D analogs, and a combination thereof. In some embodiments, the vitamin D compound is cholecalciferol. In some embodiments, the vitamin D compound has an activity ranging from 10 IU to 6000 IU. In some embodiments, the vitamin D compound has an activity ranging from 100 IU to 4000 IU. In some embodiments, the vitamin D compound has an activity ranging from 200 IU to 2000 IU. In some embodiments, the vitamin D compound has an activity ranging from 400 IU to 1000 IU. In some embodiments, the vitamin D compound has an activity ranging from 10 IU to 200 IU.

In some embodiments, the composition has a low viscosity. In some embodiments, the composition has a viscosity of less than 10 cp, less than 9 cp, less than 8 cp, less than 7 cp, less than 6 cp, less than 5 cp, less than 4 cp, less than 3 cp, less than 2 cp, or less than 1 cp at 25° C. In some embodiments, the composition further comprises dissolved carbon dioxide. In some embodiments, the positive pressure is applied to the composition by the escape of the carbon dioxide from the composition as the temperature of the composition increases. In some embodiments, the composition is contacted with the nipple of a breast on the 2^{nd} week of the individual's menstrual cycle. In some embodiments, the composition is contacted with the nipple of a breast for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

In some embodiments, the methods further comprise applying a topical anesthetic to the nipple before the composition is contacted with the nipple. In some embodiments, the methods further comprise cleaning the nipple before the composition is contacted with the nipple. In some embodiments, the device further comprises: a first opening sized to circumscribe a nipple, which opening is operatively connected to the treatment chamber. In some embodiments, the device further comprises: a second opening operatively connected to the treatment chamber through which through which the composition is instilled into the treatment chamber. In some embodiments, the device further comprises a third opening operatively connected to the treatment chamber through which positive pressure is applied to the composition. In some embodiments, the methods further comprise adhering the device to the nipple. In some embodiments, the device further comprises an adhesive which adheres the device to the breast. In some embodiments, the adhesive is a silicone-based skin adhesive. In some embodiments, the methods further comprise applying a cover over the nipple after removing the device. In some embodiments, the cover is waterproof. In some embodiments, the cover is airtight. In some embodiments, the cover is opaque. In some embodiments, the cover comprises a liquid bandage. In some embodiments, the cover comprises a patch. In some embodiments, the cover comprises a film. In some embodiments, the cover comprises an occlusive agent. In some embodiments, the cover comprises an anti-inflammatory agent, an antioxidant, or an antiseptic.

Described herein, in certain embodiments, are methods of treating a breast disorder, comprising: contacting a treatment chamber of a device comprising a composition comprising endoxifen or a pharmaceutically acceptable salt thereof with a nipple of a breast of an individual; and applying positive pressure on the composition. In some embodiments, the composition is forced into 1 to 5 breast ducts. In some embodiments, the composition is forced into 4 to 8 breast ducts. In some embodiments, the composition is forced into 7 to 11 breast ducts. In some embodiments, the breast disorder is proliferative breast disease, breast cancer or increased breast density. In some embodiments, the proliferative breast disease is atypical ductal hyperplasia or atypical lobular hyperplasia. In some embodiments, the breast cancer is ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is ER+ breast cancer, HER2+ breast cancer, or triple-negative breast cancer. In some embodiments, the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma.

In some embodiments, the individual is tamoxifen resistant. In some embodiments, the individual has been predicted to have a (i) moderate to high risk of cancer relapse or (ii) low to moderate rate of disease-free survival. In some embodiments, the moderate or high risk of cancer relapse is due to reduced expression or reduced function of a member of cytochrome P450 family. In some embodiments, the member of cytochrome P450 family is CYP2D6, CYP2B6, CYP2C9, CYP2C19, CYP3A4, or CYP3A5. In some embodiments, the individual has immune suppression. In some embodiments, the individual has a high risk of tumor escape. In some embodiments, the individual has increased activity or expression of IDO1, IDO2, TDO, or a combination thereof.

In some embodiments, the endoxifen is an E-isomer, a Z-isomer or a mixture thereof. In some embodiments, the composition comprises endoxifen or a pharmaceutically acceptable salt thereof at a concentration ranging from 0.01% to 15% by weight of the composition. In some embodiments, the composition comprising endoxifen is formulated in a hydroalcoholic gel, a hydroalcoholic solution, a patch, a cream, an emulsion, a lotion, an ointment, a powder, a paste, or an oil. In some embodiments, the composition further comprises at least one therapeutic agent. In some embodiments, the composition further comprises a plurality of therapeutic agents. In some embodiments, the therapeutic agent is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anti-cancer antibodies, immunotherapy agents, anthracyclins, platinums, vinca alkoids, camptothecins, taxanes, hormones, 1-alpha-hydroxylase inhibitors, 24-hydroxylase inhibitors, or a combination thereof.

In some embodiments, the composition further comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, 4-hydroxytamoxifen, N-desmethyltamoxifen, endocoxifen, lasofoxifene, raloxifene, benzothiophene, bazedofoxifene, arzoxifene, miproxifene, levormeloxifene, droloxifene, clomifene, idoxifene, EM652, ERA-92, toremifene, trastuzumab, vinorelbine, butyric acid, epirubicin, doxorubicin, and combinations thereof. In some embodiments, the therapeutic agent is a tryptophan metabolism inhibitor, a kynurenine depletor, an inhibitor of IDO1, IDO2, TDO, or a combination thereof. In some embodiments, the therapeutic agent is an inhibitor of IDO1, IDO2, TDO, or a combination thereof. In some embodiments, the therapeutic agent is a PD-L1 inhibitor, a PD-1 inhibitor, a CTLA-4 inhibitor, or a combination thereof. In some embodiments, the composition further comprises at least one omega-3 fatty acid, at least one vitamin D compound or a pharmaceutically acceptable salt thereof, or a combination thereof.

In some embodiments, the composition comprises (i) endoxifen or a pharmaceutically acceptable salt thereof; (ii) at least one an omega-3 fatty acid; and (iii) at least one vitamin D compound or a pharmaceutically acceptable salt thereof. In some embodiments, the composition comprises the omega-3 fatty acid at a concentration ranging from 10% to 90% by weight of the composition. In some embodiments, the omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof. In some embodiments, the omega-3 fatty acid is a triglyceride or a phospholipid. In some embodiments, the composition further comprises a mixture of EPA and DHA. In some embodiments, the vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4, 1,25 dihydroxy-19-nor-vitamin D2, 1-alphahydroxyvitamin D3, vitamin D analogs, and a combination thereof. In some embodiments, the vitamin D compound is cholecalciferol. In some embodiments, the vitamin D compound has an activity ranging from 10 IU to 6000 IU. In some embodiments, the vitamin D compound has an activity ranging from 100 IU to 4000 IU. In some embodiments, the vitamin D compound has an activity ranging from 200 IU to 2000 IU. In some embodiments, the vitamin D compound has an activity ranging from 400 IU to 1000 IU. In some embodiments, the vitamin D compound has an activity ranging from 10 IU to 200 IU.

In some embodiments, the composition has a low viscosity. In some embodiments, the composition has a viscosity of less than 10 cp, less than 9 cp, less than 8 cp, less than 7 cp, less than 6 cp, less than 5 cp, less than 4 cp, less than 3 cp, less than 2 cp, or less than 1 cp at 25° C. In some embodiments, the composition further comprises dissolved carbon dioxide. In some embodiments, the positive pressure is applied to the composition by the escape of the carbon dioxide from the composition as the temperature of the composition increases. In some embodiments, the composition is contacted with the nipple of a breast on the 2^{nd} week of the individual's menstrual cycle. In some embodiments, the composition is contacted with the nipple of a breast for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

In some embodiments, the methods further comprise applying a topical anesthetic to the nipple before the composition is contacted with the nipple. In some embodiments, the methods further comprise cleaning the nipple before the composition is contacted with the nipple. In some embodiments, the device further comprises: a first opening sized to circumscribe a nipple, which opening is operatively connected to the treatment chamber; and a second opening operatively connected to the treatment chamber through which positive pressure is applied to the composition comprising endoxifen or a pharmaceutically acceptable salt thereof. In some embodiments, the device further comprises a third opening through which the composition comprising at least one therapeutic agent is instilled into the treatment chamber. In some embodiments, the methods further comprise adhering the device to the nipple. In some embodiments, the device further comprises an adhesive which adheres the device to the breast. In some embodiments, the methods further comprise applying a cover over the nipple after removing the device. In some embodiments, the cover is waterproof and/or airtight and/or opaque. In some embodiments, the cover comprises a liquid bandage. In some embodiments, the cover comprises a patch. In some embodiments, the cover comprises a film. In some embodiments, the cover comprises an occlusive agent. In some embodiments, the cover comprises an anti-inflammatory agent, an anti-oxidant, or an antiseptic.

Described herein, in certain embodiments, are compositions for use in the treatment of a breast disorder in an individual, comprising endoxifen or a pharmaceutically acceptable salt thereof, and a dissolved gas. In some embodiments, the dissolved gas is carbon dioxide. In some embodiments, the individual has a breast disorder. In some embodiments, the breast disorder is a proliferative breast disease, a breast cancer, or increased breast density. In some embodiments, the proliferative breast disease is atypical ductal hyperplasia or atypical lobular hyperplasia. In some embodiments, the breast cancer is ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is ER+ breast cancer, HER2+ breast cancer, or triple-negative breast cancer. In some embodiments, the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma. In some embodiments, the individual is tamoxifen resistant.

In some embodiments, the individual has been predicted to have a (i) moderate to high risk of cancer relapse or (ii) low to moderate rate of disease-free survival. In some embodiments, the moderate or high risk of cancer relapse or low to moderate rate of disease-free survival is due to a: reduced expression or reduced function of a member of cytochrome P450 family; or increased expression or an increased activity of IDO1, IDO2, TDO, or a combination thereof. In some embodiments, the member of cytochrome P450 family is CYP2D6, CYP2B6, CYP2C9, CYP2C19, CYP3A4, or CYP3A5.

In some embodiments, the individual has immune suppression. In some embodiments, the individual has a high risk of tumor escape. In some embodiments, the individual has increased activity or expression of IDO1, IDO2, TDO, or a combination thereof.

In some embodiments, the endoxifen is an E-isomer, a Z-isomer or a mixture thereof. In some embodiments, the composition comprises endoxifen or a pharmaceutically acceptable salt thereof at a concentration ranging from 0.01% to 15% by weight of the composition. In some embodiments, the composition comprising endoxifen is formulated in a hydroalcoholic gel, a hydroalcoholic solution, a patch, a cream, an emulsion, a lotion, an ointment, a powder, a paste, or an oil. In some embodiments, the composition further comprises at least one therapeutic agent. In some embodiments, the composition further comprises a plurality of therapeutic agents. In some embodiments, the therapeutic agent is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anti-cancer antibodies, immunotherapy agents, anthracyclins, platinums, vinca alkoids, camptothecins, taxanes, hormones, 1-alpha-hydroxylase inhibitors, 24-hydroxylase inhibitors, or a combination thereof. In some embodiments, the composition further comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, 4-hydroxytamoxifen, N-desmethyltamoxifen, endocoxifen, lasofoxifene, raloxifene, benzothiophene, bazedofoxifene, arzoxifene, miproxifene, levormeloxifene, droloxifene, clomifene, idoxifene, EM652, ERA-92, toremifene, trastuzumab, vinorelbine, butyric acid, epirubicin, doxorubicin, and combinations thereof. In some embodiments, the therapeutic agent is a tryptophan metabolism inhibitor, a kynurenine depletor, an inhibitor of IDO1, IDO2, TDO, or a combination thereof. In some embodiments, the therapeutic agent is an inhibitor of IDO1, IDO2, TDO, or a combination thereof. In some embodiments, the therapeutic agent is a PD-L1 inhibitor, a PD-1 inhibitor, a CTLA-4 inhibitor, or a combination thereof. In some embodiments, the composition further comprises at least one omega-3 fatty acid, at least one vitamin D compound or a pharmaceutically acceptable salt thereof, or a combination thereof.

In some embodiments, the composition comprises (i) endoxifen or a pharmaceutically acceptable salt thereof; (ii) at least one an omega-3 fatty acid; and (iii) at least one vitamin D compound or a pharmaceutically acceptable salt thereof. In some embodiments, the composition comprises the omega-3 fatty acid at a concentration ranging from 10% to 90% by weight of the composition. In some embodiments, the omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof. In some embodiments, the composition comprises a mixture of EPA and DHA. In some embodiments, the omega-3 fatty acid is a triglyceride or a phospholipid. In some embodiments, the vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4, 1,25 dihydroxy-19-nor-vitamin D2, 1-alphahydroxyvitamin D3, vitamin D analogs, and a combination thereof. In some embodiments, the vitamin D compound is cholecalciferol. In some embodiments, the vitamin D compound has an activity ranging from 10 IU to 6000 IU. In some embodiments, the vitamin D compound has an activity ranging from 100 IU to 4000 IU. In some embodiments, the vitamin D compound has an activity ranging from 200 IU to 2000 IU. In some embodiments, the vitamin D compound has an activity ranging from 400 IU to 1000 IU. In some embodiments, the vitamin D compound has an activity ranging from 10 IU to 200 IU.

In some embodiments, the composition has a low viscosity. In some embodiments, the composition has a viscosity of less than 10 cp, less than 9 cp, less than 8 cp, less than 7 cp, less than 6 cp, less than 5 cp, less than 4 cp, less than 3 cp, less than 2 cp, or less than 1 cp at 25° C. In some embodiments, the composition further comprises dissolved carbon dioxide. In some embodiments, the positive pressure is applied to the composition by the escape of the carbon dioxide from the composition as the temperature of the composition increases. In some embodiments, the composition is contacted with the nipple of a breast on the 2nd week of the individual's menstrual cycle. In some embodiments, the composition is contacted with the nipple of a breast for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours.

Described herein, in certain embodiments, are devices for delivering a composition to a breast duct of an individual in need thereof, comprising: a treatment chamber; a first opening sized to circumscribe a nipple, which opening is operatively connected to the treatment chamber; and a composition comprising endoxifen or a pharmaceutically acceptable salt thereof. In some embodiments, the composition comprising the endoxifen or a pharmaceutically acceptable salt thereof is contained within the treatment chamber. In some embodiments, the devices further comprise a second opening operatively connected to the treatment chamber through which through which the composition is instilled into the treatment chamber. In some embodiments, the devices further comprise a third opening operatively connected to the treatment chamber through which positive pressure is applied to the composition. In some embodiments, the endoxifen is an E-isomer, a Z-isomer or a mixture thereof. In some embodiments, the composition comprises endoxifen or a pharmaceutically acceptable salt thereof at a concentration ranging from 0.01% to 15% by weight of the composition. In some embodiments, the composition comprising endoxifen is formulated in a hydroalcoholic gel, a hydroalcoholic solution, a patch, a cream, an emulsion, a lotion, an ointment, a powder, a paste, or an oil. In some embodiments, the composition further comprises at least one therapeutic agent. In some embodiments, the composition comprises a plurality of therapeutic agents.

In some embodiments, the therapeutic agent is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anti-cancer antibodies, immunotherapy agents, anthracyclins, platinums, vinca alkoids, camptothecins, taxanes, hormones, 1-alpha-hydroxylase inhibitors, 24-hydroxylase inhibitors, or a combination thereof. In some embodiments, the composition further comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, 4-hydroxytamoxifen, N-desmethyltamoxifen, endocoxifen, lasofoxifene, raloxifene, benzothiophene, bazedofoxifene, arzoxifene, miproxifene, levormeloxifene, droloxifene, clomifene, idoxifene, EM652, ERA-92, toremifene, trastuzumab, vinorelbine, butyric acid, epirubicin, doxorubicin, and combinations thereof. In some embodiments, the therapeutic agent is a tryptophan metabolism inhibitor, a kynurenine depletor, an inhibitor of IDO1, IDO2, TDO, or a combination thereof. In some embodiments, the therapeutic agent is an inhibitor of IDO1, IDO2, TDO, or a combination thereof. In some embodiments, the therapeutic agent is a PD-L1 inhibitor, a PD-1 inhibitor, a CTLA-4 inhibitor, or a combination thereof. In some embodiments, the composition further comprises at least one omega-3 fatty acid, at least one vitamin D compound or a pharmaceutically acceptable salt thereof, or a combination thereof.

In some embodiments, the composition further comprises (i) endoxifen or a pharmaceutically acceptable salt thereof; (ii) at least one an omega-3 fatty acid; and (iii) at least one vitamin D compound or a pharmaceutically acceptable salt thereof. In some embodiments, the composition comprises the omega-3 fatty acid at a concentration ranging from 10% to 90% by weight of the composition. In some embodiments, the omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof. In some embodiments, the composition comprises a mixture of EPA and DHA. In some embodiments, the omega-3 fatty acid is a triglyceride or a phospholipid. In some embodiments, the vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4, 1,25 dihydroxy-19-nor-vitamin D2, 1-alphahydroxyvitamin D3, vitamin D analogs, and a combination thereof. In some embodiments, the vitamin D compound is cholecalciferol. In some embodiments, the vitamin D compound has an activity ranging from 10 IU to 6000 IU. In some embodiments, the vitamin D compound has an activity ranging from 100 IU to 4000 IU. In some embodiments, the vitamin D compound has an activity ranging from 200 IU to 2000 IU. In some embodiments, the vitamin D compound has an activity ranging from 400 IU to 1000 IU. In some embodiments, the vitamin D compound has an activity ranging from 10 IU to 200 IU.

In some embodiments, the composition has a low viscosity. In some embodiments, the composition has a viscosity of less than 10 cp, less than 9 cp, less than 8 cp, less than 7 cp, less than 6 cp, less than 5 cp, less than 4 cp, less than 3 cp, less than 2 cp, or less than 1 cp at 25° C. In some embodiments, the composition further comprises dissolved carbon dioxide. In some embodiments, the positive pressure is applied to the composition by the escape of the carbon dioxide from the composition as the temperature of the composition increases. In some embodiments, the composition is contacted with the nipple of a breast on the 2^{nd} week of the individual's menstrual cycle. In some embodiments, the composition is contacted with the nipple of a breast for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some embodiments, delivering a composition to the breast duct of the individual in need thereof further comprises applying a topical anesthetic to the nipple before the composition is contacted with the nipple. In some embodiments, delivering a composition to the breast duct of the individual in need thereof further comprises cleaning the nipple before the composition is contacted with the nipple. In some embodiments, delivering a composition to the breast duct of the individual in need thereof further comprises adhering the device to the nipple. In some embodiments, delivering a composition to the breast duct of the individual in need thereof further comprises an adhesive which adheres the device to the breast.

Described herein, in certain embodiments, are kits for delivering a composition to a breast duct of an individual in need thereof, comprising: a device for delivering a composition to a breast duct of the individual; a composition comprising endoxifen or a pharmaceutically acceptable salt thereof; and instructions for use of the device and the composition. In some embodiments, the device further comprises a treatment chamber comprising the composition. In some embodiments, the composition further comprises at least one therapeutic agent. In some embodiments, the composition further comprises a plurality of therapeutic agents.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Current best practice for the treatment of breast cancer is to diagnose breast cancer with mammography and then to cut, bum, and treat the patient using extreme methods, such as surgery, radiation therapy, and chemotherapy. Surgery and radiation are local, but chemotherapy is systemic.

Systemic chemotherapy is accompanied by often severe side effects. These side effects include, but are not limited to, hair loss, mouth sores, nausea and vomiting, neutropenia, premature menopause, infertility, neuropathy, cardiomyopathy, Hand-foot syndrome, myelodysplastic syndrome, and acute myeloid leukemia.

Proliferative breast disease (PBD), including ductal hyperplasia, lobular hyperplasia, atypical ductal hyperplasia, atypical lobular hyperplasia, ductal carcinoma in situ, and lobular carcinoma, is difficult to diagnosis by current imaging methods because it involves such small numbers of cells that even the most modem imaging methods fail to detect it.

Although mammography generally reduces the number of deaths from cancer among women ages 40 to 74, it has several drawbacks, including: false-positive results and over-diagnosis, false-negative results and under diagnosis, and radiation exposure, likely due to erroneous assignment of the breast lesions by radiologists into BI-RADS categories III and IV. One of the contributing factors for erroneous BI-RADS assignment is that many of the individuals undergoing mammography have dense breasts. PBD and breast cancer are often masked by the presence of dense breast during mammography scanning. While there is a high degree of concordance and agreement in assignment by radiologists of breast lesions into the categories 0 to II, and into categories V and VI, the degree of concordance is low in the assignment of final assessment BI-RADS categories III and IV due to a higher degree of erroneous assignment by the radiologists.

With respect to treatment, local, effective, and easy-to-administer therapy would make early diagnosis possible and obviate the side effects of systemic treatment and could produce higher levels of drugs in the breast, improving efficacy.

Intraductal treatment with pharmaceuticals has been shown to be effective, even with very little drug reaching the blood stream, which reduces side effects. However, cannulating the correct duct can be a challenge, and it can cause considerable pain.

Transdermal treatment with topical formulations are promising, however the delivery of such transdermal compositions can be limited due to the barrier functions performed by the skin. Although inclusion of some permeation enhancers can mitigate some of the limitations, improved methods of drug delivery would be beneficial. The barrier function of skin is usually performed by stratified keratinocytes known as comeocytes. Comeocytes of the mammary papilla (breast areolae and nipple) epidermis are smaller in size and less concentrated compared with the comeocytes in the skin on rest of the body (US2014/0088059; Kikuchi et al. Br. J. Dermatology, 2011, 164, pages 97-102). Additionally, the number of layers of comeocyte cells is lower in the mammary papillae, having 14 cell layers, compared to the adjacent breast skin, having 17 cell layers. As a result, the epidermis of the mammary papillae is more permeable than normal skin. Further, the surface lipid levels are higher in the areolae affecting the hydration levels of the skin. (Id.). Thus, transpapillary methods for local administration of drugs, particularly those that have poor aqueous solubility, to the breast areolae and nipples is very attractive.

Transpapillary methods have been developed using iontophoresis. These methods involve application of an electric current to the breast that “conducts” a drug into the ducts of the breast. This method often results in discomfort to the patient and is limited to drugs which have a net charge.

Passive, transpapillary methods have been tried and a recent publication has demonstrated the feasibility of drug permeation into the mammary papillae using cadaver skin models, but to date there have been no studies to demonstrate these would be efficacious in humans, (Transpapillary Drug Delivery to the Breast. Dave, K. et. Al, (2014) PLoS ONE 9(12): e115712, doi:10.1371/joumal.pone.0115712; U.S. Pat. Pub. No. 20140088059).

There exists an unmet need for a locally acting medicament for the diagnosis and treatment of breast conditions.

Disclosed herein, in certain embodiments, are methods of delivering a composition to a breast duct of an individual in need thereof, comprising: (a) contacting a composition comprising endoxifen or a pharmaceutically acceptable salt thereof, contained within a treatment chamber of a device with a nipple of a breast; and (b) applying positive pressure on the composition. Disclosed herein, in certain other embodiments, are methods of delivering a composition to a breast duct of an individual in need thereof, comprising: (a) contacting a composition comprising inhibitors of kynurenine pathway or a pharmaceutically acceptable salt thereof, contained within a treatment chamber of a device with a nipple of a breast; and (b) applying positive pressure on the composition. Kynurenine pathway, also known as the IDO pathway is implicated in the progression of cancers and T-regulatory cells mediated immune suppression observed in cancer patients. Accordingly, in some embodiments, the inhibitors of kynurenine pathway are inhibitors of IDO1, IDO2, TDO, or a combination thereof. In some embodiments, compositions that are delivered to a breast duct of an individual in need thereof include endoxifen or a pharmaceutically acceptable salt thereof, inhibitors of the kynurenine pathway, kynurenine depletors, and inhibitors of IDO1, IDO2, TDO, or a combination thereof.

In some embodiments, the composition is forced into the breast duct due to the positive pressure. In some embodiments, the composition is forced into one or more breast ducts. In some embodiments, the composition is forced into 2 to 5 breast ducts. In some embodiments, the composition is forced into 4 to 8 breast ducts. In some embodiments, the composition is forced into 7 to 11 breast ducts.

In one aspect, the device further comprises: a first opening sized to circumscribe a nipple, which opening is operatively connected to the treatment chamber. In some embodiments, the device further comprises: a second opening operatively connected to the treatment chamber through which through which the composition is instilled into the treatment chamber. In some embodiments, the device further comprises a third opening operatively connected to the treatment chamber through which positive pressure is applied to the composition. In some embodiments, the composition comprises endoxifen or a pharmaceutically acceptable salt thereof.

In some embodiments, the compositions disclosed herein further comprise at least one therapeutic agent. In some embodiments, the compositions further comprise a plurality of therapeutic agents. In some embodiments, the composition has a low viscosity. In some embodiments, the composition has a viscosity of less than 10 cp, less than 9 cp, less than 8 cp, less than 7 cp, less than 6 cp, less than 5 cp, less than 4 cp, less than 3 cp, less than 2 cp, or less than 1 cp at 25° C. In some embodiments, the composition comprises dissolved carbon dioxide. In some embodiments, the composition is stored between 0° C. and 20° C. In some embodiments, the positive pressure is applied to the composition by the escape of the carbon dioxide from the composition as the temperature of the composition increases. In some embodiments, the composition is contacted with the nipple of a breast on the 2nd week of the individual's menstrual cycle. In some embodiments, the composition is contacted with the nipple of a breast for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some embodiments, the methods further comprise adhering the device to the nipple. In some embodiments, the device further comprises an adhesive which adheres the device to the breast. In some embodiments, the methods further comprise cleaning the nipple before the medicament is contacted with the nipple. In some embodiments, the methods further comprise applying a cover over the nipple after removing the device. In some embodiments, the cover is waterproof and/or airtight. In some embodiments, the cover is a liquid bandage. In some embodiments, the cover is a patch. In some embodiments, the cover comprises an anti-inflammatory agent, an anti-oxidant, or an antiseptic.

Methods disclosed herein are particularly useful for the treatment of individuals having a breast disorder. Disclosed herein, in certain embodiments, are methods of treating a breast disorder, comprising: (a) contacting any of the compositions disclosed herein contained within a treatment chamber of a device with a nipple of a breast; and (b) applying positive pressure on the composition. In some embodiments, compositions useful for the treatment of breast disorders include endoxifen or a pharmaceutically acceptable salt thereof, inhibitors of the kynurenine pathway, kynurenine depletors, and inhibitors of IDO1, IDO2, TDO, or a combination thereof.

In some embodiments, the breast disorder is proliferative breast disease, breast cancer, or increased breast density. In other embodiments, the proliferative breast disease is ductal hyperplasia, lobular hyperplasia, atypical ductal hyperplasia, or lobular hyperplasia. In some embodiments, the breast cancer is ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is ER+ breast cancer, HER2+ breast cancer, or triple-negative breast cancer. In some embodiments, the breast cancer is adenoid cystic (or adenocystic) carcinoma, lowgrade adenosquamous carcinoma, medullary carcinoma, mucinous (or colloid) carcinoma, papillary carcinoma, tubular carcinoma, metaplastic carcinoma, or micropapillary carcinoma. In some embodiments, the breast disorder is increased breast density.

Increased expression and activity of tryptophan degrading enzymes, IDO1 (rate limiting enzyme), IDO2, and TDO resulting increased kynurenine levels have been shown in cancers (and host immune cells such as macrophages and dendritic cells), for e.g., in lymph nodes and has been suggested to aid in increase immune tolerance by mediating suppressive effects on effect T cells, and recruiting and activating suppressive population of regulatory T cells (T-regs). IDO pathway has thus been implicated in tumor escape and metastasis of cancer, for e.g., in triple negative cancer cells as well as in immune suppression. High expression of IDO1 is also associated with poor prognosis and decreased disease-free survival in various cancer types.

Methods disclosed herein are also useful for the treatment of individuals who: (a) are tamoxifen resistant; (b) are predicted to have moderate to high risk of cancer relapse; (c) are predicted to have low to moderate rate of disease-free survival, (d) are categorized as having a BIRADS category III or a BI-RADS category IV breast lesion; (e) have hot flashes; (f) have increased expression or activity of IDO1, IDO2, TDO, or a combination thereof in (i) breast tissues, or (ii) lymph nodes (including, without limitation, sentinel lymph nodes) or both.

### Breast Disorders

As used herein, “breast disorder” means any disorder of a breast. Breast disorders include benign lesions of the breast (proliferative breast disease), breast cancer, and breast density. Benign breast lesions include, but are not limited to, ductal hyperplasia, lobular hyperplasia, atypical ductal hyperplasia, and atypical lobular hyperplasia.

As used herein, “breast cancer” means any malignant tumor of breast cells. There are several types of breast cancer. Exemplary breast cancers include, but are not limited to, ductal carcinoma in situ, lobular carcinoma in situ, invasive (or infiltrating) ductal carcinoma, invasive (or infiltrating) lobular carcinoma, inflammatory breast cancer, triple-negative breast cancer, ER+ breast cancer, HER2+ breast cancer, adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, and micropapillary carcinoma. A single breast tumor can be a combination of these types or be a mixture of invasive and in situ cancer.

Ductal hyperplasia is hyperplasia of a breast duct, not accompanied by histomorphologic abnormalities. Ductal hyperplasia is not usually considered predicative of a predisposition for breast cancer.

Lobular hyperplasia is hyperplasia of a breast lobule, not accompanied by histomorphologic abnormalities. Lobular hyperplasia is not usually considered predicative of a predisposition for breast cancer.

Atypical ductal hyperplasia (ADH) is a benign lesion of the breast characterized by hyperplasia of at least one breast duct and histomorphologic abnormalities. While not cancerous, ADH can be indicative of a predisposition for breast cancer. ADH can be excised by lumpectomy.

Atypical lobular hyperplasia is a benign lesion of the breast characterized by hyperplasia of a breast lobule and histomorphologic abnormalities. While not cancerous, ADH can be indicative of a predisposition for breast cancer. ADH can be excised by lumpectomy.

Ductal carcinoma in situ (DCIS) is the most common non-invasive breast cancer. It involves the cells lining the breast ducts. In DCIS, the cells have not spread beyond the walls of the ducts into the surrounding breast tissue. About 1 in 5 new breast cancer cases will be DCIS. DCIS is often treated by surgery to excise the cancerous tissue, and radiation therapy. In addition, chemotherapy (e.g., tamoxifen) can be used to treat DCIS.

Lobular carcinoma in situ is a pre-cancerous neoplasia. It can be indicative of a predisposition for invasive cancer. LCIS only accounts for about 15% of the in situ (ductal or lobular) breast cancers. Lobular carcinoma in situ is often treated with tamoxifen.

Invasive Ductal Carcinoma (IDC) is the most common invasive breast cancer. As the name implies, it is carcinoma that began in the breast ducts and then invaded the surrounding fatty tissue. About 8 of 10 invasive breast cancers are infiltrating ductal carcinomas. IDC is often treated by surgery to excise the cancerous tissue, and radiation therapy. In addition, chemotherapy (e.g., tamoxifen and trastuzumab) is often used to treat IDC. If the tumor is larger than 4 cm, a radial mastectomy can be performed.

Invasive lobular carcinoma (ILC) is a cancer that develops in the lobules of the breast and has invaded the surrounding tissue. About 1 invasive breast cancer in 10 is an ILC. ILC is treated by surgery to excise the cancerous tissue, and radiation therapy. In addition, chemotherapy (e.g., tamoxifen and trastuzumab) is often used as an adjuvant therapy to treat IDC.

Inflammatory breast cancer accounts for about 1% to 3% of all breast cancers. In inflammatory breast cancer, cancer cells block lymph vessels in the skin resulting in the breast turning read and feeling warm. The affected breast can become larger or firmer, tender, or itchy. Inflammatory breast cancer can be difficult to diagnose and is treated with chemotherapy, radiation therapy, and in some cases surgery.

ER+ breast cancer is characterized by the presence of estrogen receptors on the surface of the cancerous cells. Growth of ER+ cancer cells is associated with the availability of estrogen. Treatment options for ER+ breast cancer chemotherapeutic agents that block estrogen (e.g. tamoxifen).

HER2+ breast cancers are characterized by an excess of HER2 on the cell surface of the cancerous cells. HER2+ cancer is often treated with trastuzumab in combination with additional chemotherapeutic agents.

Triple-negative breast cancer is a breast cancer characterized by cells which lack estrogen receptors and progesterone receptors, and do not have an excess of the HER2 protein on their surfaces. Triple-negative breast cancers are often more invasive than other breast cancers. Because the tumor cells lack estrogen and progesterone receptors, hormone therapy (e.g., tamoxifen) is not effective. Additionally, as the cells lack the HER2 protein, drugs that target HER2 (e.g., trastuzumab) are ineffective.

Dense breasts have more gland tissue that makes and drains milk and stroma, and can often mask the presence of the early stages of breast cancer and/or proliferative disease. Breast density is an independent risk factor for developing breast cancer. Reduction of breast density can aid not only in reducing the risk of developing breast cancer but also in the improved detection by mammography of early stages of breast cancer.

### Method

In one aspect, disclosed herein, in certain embodiments, are methods of delivering a composition comprising endoxifen or a pharmaceutically acceptable salt thereof to a breast duct of an individual in need thereof, comprising: (a) contacting the composition contained within a treatment chamber of a device with a nipple of a breast; and (b) applying positive pressure on the composition.

Disclosed herein, in certain other embodiments, are methods of delivering a composition comprising an inhibitor of kynurenine pathway or a pharmaceutically acceptable salt thereof to a breast duct of an individual in need thereof, comprising: (a) contacting the composition contained within a treatment chamber of a device with a nipple of a breast; and (b) applying positive pressure on the composition. In some preferred embodiments, the inhibitors of kyn pathway inhibit enzymes IDO1, IDO2, TDO, or a combination thereof, or a pharmaceutically acceptable salt thereof. Accordingly, in some embodiments, disclosed herein, in certain embodiments, are methods of delivering a composition comprising an inhibitor of IDO1, IDO2, TDO, or a combination thereof, or a pharmaceutically acceptable salt thereof to a breast duct of an individual in need thereof, comprising: (a) contacting a composition contained within a treatment chamber of a device with a nipple of a breast; and (b) applying positive pressure on the composition.

In some embodiments, the composition is forced into the breast duct due to the positive pressure. In some embodiments, the composition is forced into one or more breast ducts. In some embodiments, the composition is forced into 2 to 5 breast ducts. In some embodiments, the composition is forced into 4 to 8 breast ducts. In some embodiments, the composition is forced into 7 to 11 breast ducts.

Disclosed herein, in certain embodiments, are methods of treating a breast disorder, comprising: (a) contacting a composition comprising endoxifen or a pharmaceutically acceptable salt thereof, contained within a treatment chamber of a device with a nipple of a breast; and (b) applying positive pressure on the composition.

Disclosed herein, in certain embodiments, are methods of treating a breast disorder or immune suppression or both, comprising: (a) contacting a composition comprising inhibitors of kynurenine pathway or a pharmaceutically acceptable salt thereof, contained within a treatment chamber of a device with a nipple of a breast; and (b) applying positive pressure on the composition.

In certain preferred embodiments, are methods of treating a breast disorder or immune suppression or both, comprising: (a) contacting a composition comprising inhibitors of IDO1, IDO2, TDO, or a pharmaceutically acceptable salt thereof, contained within a treatment chamber of a device with a nipple of a breast; and (b) applying positive pressure on the composition. In certain embodiments, are methods of treating a breast disorder, comprising: (a) contacting a composition comprising kynurenine depletors, or a pharmaceutically acceptable salt thereof, contained within a treatment chamber of a device with a nipple of a breast; and (b) applying positive pressure on the composition.

In some embodiments, the breast disorder is proliferative breast disease, breast cancer, or increased breast density. In some embodiments, the benign breast lesion or proliferative breast disease is ductal hyperplasia, lobular hyperplasia, atypical ductal hyperplasia, or atypical lobular hyperplasia. In some embodiments, the breast cancer is ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is ER+ breast cancer, HER2+ breast cancer, or triple-negative breast cancer. In some embodiments, the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma. In some embodiments, the breast disorder is increased breast density.

Methods disclosed herein are also useful for the treatment of individuals who: (a) are tamoxifen resistant; (b) are predicted to have moderate to high risk of cancer relapse; (c) are predicted to have low to moderate rate of disease-free survival, (d) are categorized as having a BIRADS category III or a BI-RADS category IV breast lesion; (e) have hot flashes; (f) have increased expression or activity of IDO1, IDO2, TDO, or a combination thereof in (i) breast tissues, or (ii) lymph nodes (including, without limitation, sentinel lymph nodes) or both.

In some embodiments, the composition is instilled into the treatment chamber by injecting it through the second opening (e.g., via a syringe operatively connected to the opening, for example via a luer system). In some embodiments, the composition comprises a therapeutic agent. In some embodiments, the composition comprises a plurality of therapeutic agents. In at least one embodiment, the composition further comprises a diagnostic agent, such as radiocontrast agents, MRI contrast agents radionuclides, and ultrasound contrast agents. Such diagnostic agents are advantageous in enabling visualization of the breast structures when such compositions are delivered to the individual and permit monitoring of the patient response to the treatment. Accordingly, the methods disclosed herein are useful in tracking and monitoring the effectiveness of the treatment and the progression (or lack of thereof) of the breast disorder.

In some embodiments, positive pressure is applied to the composition. In some embodiments, the positive pressure is applied to the composition by introducing a gas into the treatment chamber (e.g., via a syringe operatively connected to the opening, for example via a luer system). In some embodiments, the positive pressure is applied to the composition by the escape of carbon dioxide from the composition as the temperature of the composition increases.

In some embodiments, the composition is contacted with the nipple of a breast according a predetermined schedule for the composition. As the therapeutic agent is being administered topically, the therapeutically effective amounts of the compositions disclosed herein are well known to the skilled artisan who can determine an appropriate dosage schedule for the composition. In some embodiments, the composition is contacted with the nipple of a breast on the 2^{nd} week of a female individual's menstrual cycle.

In some embodiments, the composition is contacted with the nipple of a breast for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. In some embodiments, the composition is contacted with the nipple of a breast overnight. In some embodiments, the composition is contacted with the nipple daily, weekly, biweekly, semimonthly, monthly, quarterly, 6 monthly or yearly as determined by the attending physician.

In some embodiments, the method further comprises anesthetizing the nipple. In some embodiments, the nipple is contacted with a topical anesthetic. In some embodiments, the topical anesthetic comprises lidocaine. In some embodiments, the topical anesthetic is EMLA Cream (lidocaine 2.5% and prilocaine 2.5%), or Topicaine (4% lidocaine or 5% lidocaine).

In some embodiments, the methods further comprise cleaning the nipple before the composition is contacted with the nipple. The nipple is cleaned by any suitable method. In some embodiments, the nipple is sterilized. In some embodiments, debris (e.g., keratin plugs) is removed from the nipple, increasing access to ducts of the nipple. In some embodiments, the nipple is scrubbed with a mild scrub with a dekeratinizing gel. In some embodiments, the nipple is scrubbed with an exfoliant. Any suitable exfoliant can be used with the methods disclosed herein. Examples of suitable exfoliants include, but are not limited to, microfiber cloths, adhesive exfoliation sheets, micro-bead facial scrubs, crepe paper, crushed apricot kernel or almond shells, sugar or salt crystals, pumice, and abrasive materials such as sponges, loofahs, brushes, salicylic acid, glycolic acid, fruit enzymes, citric acid, malic acid, alpha hydroxy acids (AHAs), and beta hydroxy acids (BHAs). In some embodiments, cleaning the nipple results in the opening of ducts of the nipple. In some embodiments, the ducts of a nipple are about 0.1 to about 0.3 mm in diameter after cleaning.

In some embodiments, the methods further comprise applying a cover over the nipple after removing the device. In some embodiments, the cover is waterproof and/or airtight and/or opaque (light-tight). In some embodiments, the cover comprises a liquid bandage. In some embodiments, the cover comprises a wound dressing, e.g., a bandage or a patch. In some embodiments, the cover comprises a film. In some embodiments the cover comprises an occlusive agent (e.g., petroleum jelly, mineral oil, shea butter, lanolin, paraffin, beeswax, squalene, triglycerides, coconut oil, sunflower oil, sesame oil, soybean oil, jojoba oil, evening primrose oil and olive oil). In some embodiments, the cover comprises an anti-inflammatory agent or an antiseptic agent.

In one aspect, the methods comprise screening individuals for tamoxifen resistance. An individual is classified as “tamoxifen resistant” if the individual is an intermediate or a poor metabolizer of tamoxifen. Cytochrome P450 (CYP) enzymes, including CYP2D6, metabolize tamoxifen resulting in the formation of metabolites 4-hydroxytamoxifen and endoxifen. More than one hundred CYP2D6 alleles are known in the art resulting into four phenotypes: ultra-rapid metabolizers, extensive metabolizers, intermediate metabolizers and poor metabolizers based on CYP2D6 enzyme activity and the levels of endoxifen in the blood. Patients stratified genetically into CYP2D6 intermediate or poor metabolizers showed gene-dose dependent decrease in the formation of endoxifen plasma concentrations compared to extensive metabolizers.

Accordingly, in some embodiments, the methods comprise determining if an individual's is tamoxifen resistant prior to delivery of a composition comprising endoxifen or a pharmaceutically acceptable salt thereof to a breast duct of the individual. Accordingly, in some embodiments, the methods comprise collecting a biological sample from an individual, and analyzing the sample for presence of tamoxifen metabolite endoxifen. The biological sample can be any sample that permits analysis of the individual's proteins, peptides, polypeptides, nucleotides, polynucleotides, DNA, mRNA, genes etc., and includes, without limitation, the individual's cells, tissues, blood, plasma, serum, ductal fluids, circulating microvessicles, etc. If the levels of endoxifen are low or poor, such individuals would be classified as tamoxifen resistant.

In some embodiments, the biological samples can also be analyzed for the presence of gene variants of cytochrome P450 family members such as CYP2D6, CYP2C9, CYP2C19, CYP2B6, CYP3A4, CYP3A5, and the like. For example, the presence of CYP2D6 “null” alleles (*4, *5, *5-*8, *11-*16, *18-*21, *36, *38, *40, *42* 44, *56, and *62) or “duplicated” alleles (*4×N, *6×N, and *36×N) would indicate the individual is a poor metabolizer of tamoxifen and would benefit from localized delivery of a composition comprising endoxifen or a pharmaceutically acceptable salt thereof to the affected breast duct.

The presence of CY2D6 “reduced activity” alleles (*9, *10, *17, *29, *41, and *59) or “duplicated” alleles (*10×N, *17×N, *29×N, and *41×N) would indicate that the individual is an intermediate metabolizer of tamoxifen and would also benefit from localized delivery of a composition comprising endoxifen or a pharmaceutically acceptable salt thereof to the affected breast duct.

Similarly, presence of CYP3A *22 would indicate that the individual is likely a poor tamoxiphen metabolizer and will have low blood endoxifen levels upon tamoxifen treatment, and would therefore benefit from localized delivery of a composition comprising endoxifen or a pharmaceutically acceptable salt thereof to the affected breast duct.

Treating tamoxifen resistant individual's with localized delivery of a composition comprising endoxifen or a pharmaceutically acceptable salt thereof to the affected breast duct would bypassing the CYP mediated degradation of tamoxifen and the low in vivo endoxifen production

Further, women who take tamoxifen have a 2- to 3-fold higher risk of experiencing hot flashes compared to women who do not take tamoxifen. Therefore, physicians often prescribe selective serotonin reuptake inhibitor (SSRI) antidepressants, such as venlaflaxine, paroxetine and fluoxetine, for managing hot flashes. However, some women who are on certain SSRis, e.g., paroxetine and fluoxetine, have reduced efficacy of treatment with tamoxifen, mainly due to drug interactions. Paroxetine and fluoxetine are known inhibitors of cytochrome P450 enzymes, (e.g., CYP2D6, and CYP3A4), the rate-limiting enzymes in tamoxifen metabolism and such inhibition is dependent on the type of gene variant present in the individual. On the other hand, SSRis such as venlaflaxine, sertraline, citalopram, escitalopram, and fluvoxamine are weak inhibitors of CYP2D6. Indeed, variants of CYP2D6 (for e.g., CYP2D6*4, *3, *5, *6 variants) and CYP3A4/5 genotypes have been shown to be associated with altered endoxifen levels in patients.

Thus, determination of the individual's CYP genotype would be advantageous in determining the treatment regimen and disease management. Individuals with breast disorders and who have CYP gene variants that are related to tamoxifen resistance and/or drug interactions with other drugs such as SSRI would benefit from (i) localized delivery of a composition comprising endoxifen or a pharmaceutically acceptable salt thereof to the affected breast duct bypassing the CYP mediated endoxifen in vivo production; or (ii) determining the CYP genotype of the individual and based on the genotype, undergo hot flash management with a different SSRI; or (iii) both.

Accordingly, in some embodiments, the methods disclosed herein comprise determining an individual's potential for drug interaction and response to selective serotonin reuptake inhibitor (SSRI) treatment and tamoxifen. It would be advantageous for a prescribing physician to be able to select an appropriate drug, e.g., SSRI for an individual when on tamoxifen or tamoxifen metabolite therapy based on the individual's CYP profile. In some embodiments, the methods comprise collecting a biological sample from the individual and determining the presence of gene variants of cytochrome P450 family members such as CYP2D6, CYP2C9, CYP2C19, CYP2B6, CYP3A4, CYP3A5 and the like.

Gene analyses can be conducted by any of the methods known in the art, and gene analyses include, without limitation, genotyping, sequencing, restriction fragment length polymorphism (RFLP), single nucleotide polymorphism, mutations (including deletions, insertions, inversions, duplications) etc. For these analyses, PCR, sequencing, hybridization to arrays, microfluidics etc. can be used. Analytical methods include without limitation, di-deoxy sequencing, next generation sequencing, whole genome sequencing, exome mapping, transcriptome mapping etc.

In some embodiments, the methods comprise determining an individual's kynurenine levels and/or expression of IDO1, IDO2, and/or TDO enzymes in breast tissue, lymph nodes (including sentinel lymph nodes) or both as a biomarker for cancer progression and for prognosis for breast cancer and disease-free survival. High expression of any of these enzymes, particularly IDO1, will be indicative of a poor prognosis for breast cancer, high risk of cancer relapse and/or low to moderate rate of disease-free survival. In other embodiments, the methods comprise determining the individual's kynurenine levels in ductal fluid. Assays to determine enzyme expression and activity are known in the art (Bubnoff et al. J. Immunol. 2011, vol. 186(12), pages 6701-6709; Braun et al. Blood, 2005, vol. 106(7), pages 2375-2381).

### Device

Disclosed herein, in certain embodiments, are methods of delivering a composition disclosed herein to a breast duct of an individual in need thereof, comprising: (a) contacting a composition contained within a treatment chamber of a device with a nipple of a breast; and (b) applying positive pressure on the composition.

The device is constructed of any suitable material. In some embodiments, the device is made of a rigid material. In some embodiments, the device is made of a flexible material. In some embodiments, the device is made of a rigid plastic. In some embodiments, the device is made of a flexible plastic. Any FDA approved material can be used with the devices disclosed herein. In some embodiments, the device is transparent.

In some embodiments, the device comprises a treatment chamber. In some embodiments, the treatment chamber is a hollow receptacle. The treatment chamber is any suitable shape or size which will allow it to operatively cover a nipple of a breast.

The treatment chamber is sized such that it is able to cover a nipple and hold between about 0.5 cc and 10 cc of a composition described herein. In some embodiments, the treatment chamber is sized such that it is able to cover a nipple and hold between about 0.5 cc and 5 cc of a composition described herein. In some embodiments, the treatment chamber is sized such that it is able to cover a nipple and hold between about 0.5 cc and 4 cc of a composition described herein. In some embodiments, the treatment chamber is sized such that it is able to cover a nipple and hold between about 0.5 cc and 3 cc of a composition described herein. In some embodiments, the treatment chamber is sized such that it is able to cover a nipple and hold between about 0.5 cc and 2 cc of a composition described herein. In some embodiments, the treatment chamber is sized such that it is able to cover a nipple and hold about 1 cc and 2 cc of a composition described herein.

In addition to being sized in order to hold a therapeutically-effective volume of the desired composition, in some embodiments, the treatment chamber is sized such that it is able to contain a sufficient volume of headspace (ullage) which can be filled with a sufficient volume of a desired gas, for example, to increase the positive pressure on the composition.

In some embodiments, the device further comprises: a first opening sized to operative cover (or, circumscribe) a nipple, which opening is operatively connected to the treatment chamber. In some embodiments, the first opening is has any shape that is suitable for placement over a nipple. In some embodiments, the first opening is circular in shape. In some embodiments, the first opening allows the treatment chamber to be placed over and in operative contact with a nipple. The inner shape of the first opening does not need to be the same as the outer shape of the opening.

In some embodiments, the first opening is sized such that it circumscribes all or part of an areola or a nipple. In some embodiments, the first opening has a diameter of less than or about 50 mm. In some embodiments, the first opening has a diameter of less than or about 40 mm. In some embodiments, the first opening has a diameter of less than or about 30 mm. In some embodiments, the first opening has a diameter of less than or about 25 mm. In some embodiments, the first opening has a diameter of less than or about 20 mm. In some embodiments, the first opening has a diameter of less than or about 15 mm. In some embodiments, the first opening has a diameter of about 10 mm.

In at least one preferred embodiment, the device for delivering a composition to a breast duct of an individual in need thereof, comprises: (a) a treatment chamber; (b) a first opening sized to circumscribe a nipple, which opening is operatively connected to the treatment chamber; and (c) a composition comprising endoxifen or a pharmaceutically acceptable salt thereof.

In other preferred embodiments, the device for delivering a composition to a breast duct of an individual in need thereof, comprises: (a) a treatment chamber; (b) a first opening sized to circumscribe a nipple, which opening is operatively connected to the treatment chamber; and (c) a composition comprising an inhibitor of kynurenine pathway or a pharmaceutically acceptable salt thereof. In yet other preferred embodiments, the device for delivering a composition to a breast duct of an individual in need thereof, comprises: (a) a treatment chamber; (b) a first opening sized to circumscribe a nipple, which opening is operatively connected to the treatment chamber; and (c) a composition comprising an inhibitor of IDO1, IDO2, TDO, or a combination thereof, or a pharmaceutically acceptable salt thereof.

In some embodiments, the device further comprises: a second opening operatively connected to the treatment chamber through which through which the composition is instilled into the treatment chamber. In some embodiments, the second opening is a port. In some embodiments, the opening comprises a seal that inhibits or prevents backflow of the composition out of the treatment chamber. In some embodiments, the second opening is shaped such that a syringe can be operatively connected to the second opening. In some embodiments, the syringe and the second opening connect via a luer system. For example, the syringe can have a male luer lock connection fitting which is able to screw into a female luer lock fitting of the second opening, or alternatively, the syringe can have a female luer lock connection fitting which is able to screw into a male luer lock fitting of the second opening.

In some embodiments, the device further comprises a third opening operatively connected to the treatment chamber through which positive pressure is applied to the composition. In some embodiments, positive pressure is applied by filling the headspace of the treatment chamber with a gas. In some embodiments, the gas is instilled into the treatment chamber via a syringe which operatively connects to the third opening. In some embodiments, the third opening is a port. In some embodiments, the opening comprises a seal that inhibits or prevents loss the gas out of the treatment chamber. In some embodiments, the third opening is shaped such that the syringe is operatively connected to the opening. In some embodiments, the syringe and the third opening connect via a luer system. For example, the syringe can have a male luer lock connection fitting which is able to screw into a female luer lock fitting of the second opening, or alternatively, the syringe can have a female luer lock connection fitting which is able to screw into a male luer lock fitting of the third opening.

In some embodiments, the second opening allows for the installation of the composition and the application of the positive pressure (e.g., the installation of the gas). Where the second opening allows for the installation of the composition and the application of the positive pressure (e.g., the installation of the gas), a third opening can not be required.

In some embodiments, the device further comprises an adhesive which adheres the device to the breast. In some embodiments, the adhesive is any medically suitable skin adhesive. In some embodiments, the skin adhesive is applied to skin before the device is contacted with the skin. In some embodiments, the adhesive is applied to the device after the device has been contacted with the skin. In some embodiments, the adhesive creates a water tight and/or air tight seal.

In some embodiments, the adhesive secures the device to the skin for at least 24 hours. In some embodiments, the adhesive secures the device to the skin for at least 18 hours. In some embodiments, the adhesive secures the device to the skin for at least 12 hours. In some embodiments, the adhesive secures the device to the skin for at least 8 hours. In some embodiments, the adhesive secures the device to the skin for at least 6 hours.

Suitable adhesives include, but are not limited to, 2-Octyl (SecureSeal™) skin adhesive, nButyl (Liquiband®) skin adhesive, Dow Corning® 9700 Soft Skin Adhesive Parts A & B, Dow Coming® MG 7-9800 Soft Skin Adhesive Parts A & B, Dow Corning® MG 7-9850 Soft Skin Adhesive Parts A & B, Dow Corning® MG 7-9900 Soft Skin Adhesive Parts A & B. In some embodiments, the adhesive is a silicone-based skin adhesive. In some embodiments, the adhesive is a rubber-based skin adhesive. In some embodiments, the adhesive is a tape or membrane.

### Compositions

In one as aspect, disclosed herein, in certain embodiments, are methods of delivering a composition to a breast duct of an individual in need thereof, comprising: (a) contacting a composition disclosed herein contained within a treatment chamber of a device with a nipple of a breast; and (b) applying positive pressure on the composition. The compositions disclosed herein offer a way to reduce the side effects observed with the current adjuvant therapy for the treatment of breast cancer.

Without being bound by a particular theory of operation, precancerous hyperplasia of the breast is “driven” by a number of processes. A significant process is the contribution of stimulation of the estrogen/progesterone hormonal axis. Each menstrual cycle, during the proliferative phase and especially week two of the cycle, blood levels of estrogen increase significantly, driving ductal cell division and growth. Following ovulation, if fertilization does not occur, there is involution of the ductal and lobular changes and return to quiescence until the next cycle. Estrogen from systemic sources, mostly the ovaries, as well as local synthesis within the breast from the action of aromatase on testosterone contribute to the growth. A second major stimulation is the generalized effect of a pro-inflammatory environment. This has been considered by some to be the effect of stromal effects on the ductal epithelium. A third stimulation involves the role of “metabolic” drivers, such as glucose driven metabolism and high mitochondrial activity in the process. Finally, HER2 stimulation and oncogene and tumor promoter activation can contribute to either inducing hyperplasia or sustaining it.

Given the drivers of precancerous hyperplasia, certain classes of effectors can be used to reverse the hyperplasia or prevent the development or progression of breast cancer and/or metastasis. For example, estrogen receptor antagonists, like tamoxifen can block the effects of the estrogen surge. In the case of tamoxifen, it is known in the art that metabolites of tamoxifen, endoxifen and 4-hydroxytamoxifen which is 100 times more potent than tamoxifen, are likely to be the active moieties (with tamoxifen acting as a prodrug). However, it is also known in the art that certain individuals are tamoxifen resistant. Further, though tamoxifen therapy is associated with secondary benefits, improved lipid profiles and increase in bone mineral density in post-menopausal women, it has serious side effects such as rare venous thromboses and endometrial cancer, and hot flashes.

Because the risk of hot flashes is 2- to 3-fold higher among women who take tamoxifen than it is for women who do not, selective serotonin reuptake inhibitor (SSRI) antidepressants such as venlaflaxine, paroxetine and fluoxetine are often prescribed for managing hot flashes. However, some of these SSRIs (e.g., paroxetine and fluoxetine) are known to inhibit cytochrome P450 (e.g., CYP2D6, and CYP3A4) key enzymes in tamoxifen metabolism. Inhibition of the CYP2D6 reduces the levels of tamoxifen metabolite, endoxifen, which like tamoxifen also has anti-estrogenic and anti-proliferative activity. On the other hand, SSRIs such as venlaflaxine, sertraline, citalopram, escitalopram, and fluvoxamine are weak inhibitors of CYP2D6. Indeed, variants of CYP2D6 (for e.g., CYP2D6*4, *3, *5, *6 variants) and CYP3A4/5 genotypes have been shown to be associated with altered endoxifen levels in patients.

Patients stratified genetically into CYP2D6 intermediate or poor metabolizers showed genedose dependent decrease in the formation of endoxifen plasma concentrations compared to extensive metabolizers. Further, any drug that can be a substrate of CYP2D6 or CYP3A4/5 (e.g., SSRIs paroxetine and fluoxetine, antidepressants such as duloxetine and buproprion, anti-arrhythmic agents such as quinidine) that is co-prescribed with tamoxifen during adjuvant treatment resulting in poor CYP2D6 and/or CYP3A4/5 activities will also result in reduced endoxifen production and activity, and thus decrease the therapeutic benefits from tamoxifen therapy. This has been shown to increase the risk of breast cancer recurrence or cancer relapse. Thus, use of endoxifen in place of tamoxifen can bypass the metabolic steps involving CYP2D6 and/or CYP3A4 in tamoxifen resistant individuals, and in individuals at moderate to high risk for cancer relapse, or low to moderate rate of disease-free survival.

Since the metabolism of tamoxifen to active derivatives is conducted by the liver, the methods of localized administration in the instant patent teaches using tamoxifen metabolite, endoxifen, in the compositions to avoid systemic exposure. Thus, compositions comprising endoxifen or a pharmaceutically acceptable salt thereof would be particularly beneficial in tamoxifen resistant subjects. In some embodiments, compositions comprise endoxifen or pharmaceutically acceptable salt thereof. Tamoxifen resistant subjects include, without limitation, subjects with impaired activity of cytochrome P450 gene family members such as CYP2D6, CYP2C9, CYP2C19, CYP2B6, CYP3A4, CYP3A5, and ATP-binding cassette transporters such as P-glycoprotein (ABCB1) transport protein. Further, it is within the scope of this patent that the compositions comprise endoxifen wherein the endoxifen is an E-isomer, a Z-isomer, or a mixture thereof. Methods of making endoxifen are known in the art (Fauq et al. Bioorg, Med. Chem. Lett. 2010, vol. 20(10), pages 3036-3038).

The compositions comprising endoxifen or a pharmaceutically acceptable salt thereof are formulated in any form suitable for delivery through the nipple of an individual. In some embodiments, the compositions comprising endoxifen or a pharmaceutically acceptable salt thereof is formulated in a hydroalcoholic gel, a hydroalcoholic solution, a patch, a cream, an emulsion, a lotion, an ointment, a powder, a paste, or an oil. In some embodiments, the composition comprising endoxifen or a pharmaceutically acceptable salt thereof is a hydroalcoholic gel or a hydroalcoholic solution. In some embodiments, the composition comprising endoxifen or a pharmaceutically acceptable salt thereof is a an emulsion.

In another aspect, the compositions comprise inhibitors of the kynurenine pathway or pharmaceutically acceptable salts thereof. Tryptophan metabolism has been implicated in cancers, and increased levels of tryptophan metabolite, kynurenine, is a proposed biomarker for a broad variety of cancers. Increased expression and activity of the tryptophan degrading enzymes IDO1, IDO2 and TDO resulting in increased kynurenine levels has been shown in cancers, and has been suggested to aid in tumor escape (for e.g., by immune evasion and increasing immune tolerance) and malignancy as well as immune suppression due to dysregulated T-cells, particularly T-regs, the regulatory T-cells. Breast cancers show the presence of infiltration by lymphocytes. Accordingly, in some preferred embodiments, the compositions comprise an inhibitor of IDO1, IDO2, TDO, or a combination thereof. It is a particular aspect of the present invention that localized delivery of compositions comprising inhibitors of kynurenine pathway enzymes locally to breast ducts and the affected breast tissue is advantageous in affecting the surrounding stromal and T-cells locally.

Reduction of expression and/or activity of the tryptophan degrading enzymes of the kynurenine pathway (IDO1, IDO2, TDO) are also reduce the risk of invasive cancers and increase disease-free survival. A growing body of research and clinical trials with oral IDO1 inhibitors support targeting IDO1, IDO, and TDO to overcome kynurenine pathway mediated immune suppression as well as cancer escape and metastasis.

Inhibitors of the kyurenine pathway enzymes IDO1, IDO2, TDO are known in the art. IDO inhibitors can include, without limitation, i) previously established (known) IDO inhibitors, including, but not limited to: 1-methyl-DL-tryptophan (1MT; Sigma-Aldrich; St. Louis, Mo.), .beta.(3-benzofuranyl)-DL-alanine (Sigma-Aldrich), beta-(3-benzo(b)thienyl)-DL-alanine (SigmaAldrich), 6-nitro-L-tryptophan (Sigma-Aldrich), indole 3-carbinol (LKT Laboratories; St. Paul, Minn.), 3,3′-diindolylmethane (LKT Laboratories), epigallocatechin gallate (LKT Laboratories), 5-Br-4-Cl-indoxyl 1,3-diacetate (Sigma-Aldrich), 9-vinylcarbazole (Sigma-Aldrich), acemetacin (Sigma-Aldrich), 5-bromo-DL-tryptophan (Sigma-Aldrich), 5-bromoindoxyl diacetate (Sigma-Aldrich), hydroxamidine, INCB024360, epacadostat (Incyte Genetics), imidazole NLG919 (NewLink Genetics), 1-methyl-D-Tryptophan (1MT, Indoximod), and the IDO inhibitors provided in PCT2015/006520, WO2014/186035, PCT/US04/05155, PCT/US04/05154, PCT/US06/42137, U.S. patent application Ser. Nos. 11/589,024, 14/264,974; 14/322,362; 14/083,693; 14/033,117; 13/801,268; 131780510; 131777,383; 13/070069; 121736526; and U.S. patent application Pub. No. 20120277217; 20120058079, US2015175712, etc. Other IDO1, TDO inhibitors developed by pharma companies include the past and present inhbitors from Amgen, Bristol-Meyres Squibb, Curadev, Dainippon Sumitomo Pharma corp, IOmet Pharma, iTeos Therapeutics, and vertex pharmaceuticals (Rohrig, et al. J. Med. Chem. 2015, we publication May 13, 2015 incorporated by reference in its entirety). It is within the scope of the present invention that the inhibitors of kyurenine pathway enzymes include, without limitation, antibodies (monoclonal, polyclonal, hybrid, chimeric, humanized etc.), antibody fragments, conjugated antibodies, miRNA, siRNA, RNAi, small molecules, peptides, peptidomimetics, etc.

In some embodiments, the composition has a low viscosity at room temperature (between about 20° C. and 25° C.). In some embodiments, the viscosity of the composition at room temperature is suitable for transpapillary penetration.

In some embodiments, the composition has a viscosity of between about 5000 and about 0.5 cp at room temperature. In some embodiments, the composition has a viscosity of between about 2500 and about 0.5 cp at room temperature. In some embodiments, the composition has a viscosity of between about 1000 and about 0.5 cp at room temperature. In some embodiments, the composition has a viscosity of between about 750 and about 0.5 cp at room temperature. In some embodiments, the composition has a viscosity of between about 500 and about 0.5 cp at room temperature. In some embodiments, the composition has a viscosity of between about 250 cp and about 0.5 cp at room temperature. In some embodiments, the composition has a viscosity of between about 100 cp and about 0.5 cp at room temperature. In some embodiments, the composition has a viscosity of between about 50 cp and about 0.5 cp at room temperature. In some embodiments, the composition has a viscosity of between about 10 cp and about 0.5 cp at room temperature. In some embodiments, the composition has a viscosity of between about 5 cp and about 0.5 cp at room temperature. In some embodiments, the composition has a viscosity of between about 1 cp and about 0.5 cp at room temperature. In some embodiments, the composition has a viscosity of less than 10 cp, less than 9 cp, less than 8 cp, less than 7 cp, less than 6 cp, less than 5 cp, less than 4 cp, less than 3 cp, less than 2 cp, or less than 1 cp.

In some embodiments, the composition has a viscosity of less than 100 cp at room temperature. In some embodiments, the composition has a viscosity of less than 50 cp at room temperature. In some embodiments, the composition has a viscosity of less than 25 cp at room temperature. In some embodiments, the composition has a viscosity of less than 10 cp at room temperature. In some embodiments, the composition has a viscosity of less than 5 cp at room temperature. In some embodiments, the composition has a viscosity of less than 1 cp at room temperature. In some embodiments, the composition has a viscosity of less than 0.5 cp at room temperature.

### Other Therapeutic Agents

In some embodiments, the composition further comprises at least one therapeutic agent. In some embodiments, the composition further comprises a plurality of therapeutic agents.

Preventing breast cancer is possible with SERMs, SERDs, and AI, which reduce the risk of invasive disease by up to 65% (up to 73% for ER-positive and no effect for ER-negative cancer) and the risk of preinvasive disease [ductal carcinoma in situ (DCIS)] by up to 50%. Thus, in some preferred embodiments, the therapeutic agent is a SERD, a SERM, an AI, or a combination thereof. In some embodiments, the SERM is selected from the group consisting of 4-OHT, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923. In some embodiments, the SERD comprises a fulvestrant, ARN-810, or CH4986399.

While aromatase inhibitors (AI) such as are exemestane, anastrozole and letrozole are contraindicated in premenopausal women because they raise estrogen by their action in the hypothalamus, these local aromatase inhibitors in conjunction with endoxifen or Kynurenine pathway inhibitors could have synergistic effects. Thus, in some embodiments, the therapeutic agent is an aromatase inhibitor. In preferred embodiments, the therapeutic agent is exemestane, anastrozole or letrozole.

Reduction of expression and/or activity of the tryptophan degrading enzymes of the kynurenine pathway (IDO1, IDO2, TDO) are also reduce the risk of invasive cancers and increase disease-free survival. Accordingly, in some embodiments, wherein the composition comprises endoxifen or a pharmaceutically acceptable salt thereof, the therapeutic agent is an inhibitor of kynurenine pathway. Kynurenine pathway inhibitors and IDO1 inhibitors useful as therapeutic agents have been described above. Accordingly, in some embodiments, compositions comprising endoxifen or a pharmaceutically acceptable salt thereof further comprises inhibitors of kynurenine pathway. In a more preferred embodiment, the compositions comprising endoxifen or a pharmaceutically acceptable salt thereof further comprises inhibitors of IDO1, IDO2, TDO, or a combination thereof.

A growing body of work (including recent preclinical and clinical data) support targeting the HER family [epidermal growth factor receptor (EGFR), or human epidermal growth factor receptor (HER) 1 or ErbB1) and HER2, HER3, and HER4] for preventing ER-negative and possibly ER-positive breast cancer. Preclinical studies of HER family-targeting drugs in mammary neoplasia show suppression of (i) ER-negative tumors in HER2-overexpressing mouse strains, (ii) ER-tumors in mutant Brca1/p53 ρ/_mice, and (iii) ER-positive tumors in the methylnitrosourea (MNU) rat model; tumors arising in both the MNU and mutant Brca1/p53ρ/_models lack HER2 overexpression. Clinical trials include a recent placebo-controlled phase lib presurgical trial of the dual EGFR HER2 inhibitor lapatinib that suppressed growth of breast premalignancy [including atypical ductal hyperplasia (ADH) and DCIS] and invasive cancer in patients with early-stage, HER2-overexpressing or -amplified breast cancer. These results suggest that effect previously observed in a mouse model of HER2-overexpressing, ER-negative mammary cancer. Thus, in at least one embodiment, the therapeutic agent is trastuzumab.

The inflammatory target in hyperplasia is thought to be the COX-2 enzyme and therefore COX-2 inhibitors should be useful.

In some embodiments, the therapeutic agent is an anthracycline (e.g., doxorubicin or epirubicin), a platinum agent, a taxane (e.g., paclitaxel or docetaxel), or combinations thereof. In some embodiments, the therapeutic agent is ado-trastuzumab emtansine, albumin-bound paclitaxel, anastrozole, exemestane, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, 4-hydroxytamoxifen, N-desmethyltamoxifen, endocoxifen, lasofoxifene, raloxifene, benzothiophene, bazedofoxifene, arzoxifene, miproxifene, levormeloxifene, droloxifene, clomifene, idoxifene, EM652 and ERA-923, toremifene, trastuzumab, vinorelbine, or combinations thereof. In some embodiments, the therapeutic agent is tamoxifen or a tamoxifen derivative (such as 4-hydroxytamoxifen, N-desmethyltamoxifen, endoxifen and cis-tamoxifen). In some embodiments, the therapeutic agent is butyric acid. In some embodiments, the therapeutic agent is doxorubicin. In some embodiments, the therapeutic agent is epirubicin. In some embodiments, the therapeutic agent is paclitaxel. In some embodiments, the therapeutic agent is docetaxel.

In some embodiments, wherein the compositions comprise inhibitors of kynurenine pathway, the therapeutic agents are selected from the group consisting of SERDs, SERMs, AI, or a combination thereof. In some preferred embodiments, wherein the compositions comprise inhibitors of kynurenine pathway, the therapeutic agent is a tamoxifen, cis-tamoxifen, 4-hydroxytamoxifen, endoxifen, fulvestrant or anastrozole.

In other embodiments, the composition further comprises at least one omega-3 fatty acid, at least one vitamin D compound or a pharmaceutically acceptable salt thereof, or a combination thereof. In at least one embodiment, the composition comprises (i) endoxifen or a pharmaceutically acceptable salt thereof; (ii) at least one an omega-3 fatty acid; and (iii) at least one vitamin D compound or a pharmaceutically acceptable salt thereof. The concentration of the omega-3 fatty acid in the compositions disclosed herein can range between 10% to 90% by weight of the composition. In some embodiments, the concentration of the omega-3 fatty acid in the compositions disclosed herein is 10%, 20%, 30%, 40%, 50%, 60%, 70%, 80%, or 90% by weight of the composition.

As used herein, “omega-3 fatty acids” includes natural and synthetic omega-3 fatty acids, as well as pharmaceutically acceptable esters, free acids, mono-, di-, triglycerides, phospholipids, derivatives, conjugates, precursors, salts and mixtures thereof. The omega-3 fatty acid in some embodiments, is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof. In some embodiments, the composition further comprises a mixture of EPA and DHA.

In some embodiments, the omega-3 fatty acid is esterified. Non-limiting examples include alkyl esters, methyl esters, and ethyl esters. In some embodiments, the omega-3 fatty acid ester is ethyl ester. In some embodiments, the omega-3 fatty acid ester is methyl ester. In some embodiments, the omega-3 fatty acid is a triglyceride or a phospholipid. In some embodiments, triglycerides can be mono-, di-, triglycerides, or a combination thereof. In some embodiments, the triglyceride comprises same or different omega-3 acids selected from the group described above. In some embodiments, the omega-3 fatty acids of the triglycerides are short chain, medium chain, long chain fatty acids, or a combination thereof. In some embodiments, the omega-3 fatty acid is in a phospholipid form.

As used herein, “vitamin D compound” can be any vitamin D compound that can act as an active pharmaceutical ingredient and is suitable for prophylactic or therapeutic use or both, and combinations thereof, are contemplated for inclusion in the pharmaceutical composition and formulation described herein. In other embodiments, the vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4, 1,25 dihydroxy-19-nor-vitamin D2, 1-alphahydroxyvitamin D3, vitamin D analogs, and a combination thereof. In some embodiments, the vitamin D compound is cholecalciferol. In some embodiments, the vitamin D compound has an activity ranging from 10 IU to 6000 IU. In some embodiments, the vitamin D compound has an activity ranging from 100 IU to 4000 IU. In some embodiments, the vitamin D compound has an activity ranging from 200 IU to 2000 IU. In some embodiments, the vitamin D compound has an activity ranging from 400 IU to 1000 IU. In some embodiments, the vitamin D compound has an activity ranging from 10 IU to 200 IU.

In some embodiments, the therapeutic agent is a combination therapy. Where combination therapy is administered, each of the agents can be administered in combination with any other agent (e.g., simultaneously) or alone. Further, all of the agents can be administered according to the claimed method. Alternatively, some of the agents can be administered according to the claimed method, while others are administered systemically.

In some embodiments, the combination therapy is CAF: cyclophosphamide, doxorubicin, and 5-FU. In some embodiments, the combination therapy is TAC: docetaxel, doxorubicin, and cyclophosphamide. In some embodiments, the combination therapy is AC→T: doxorubicin and cyclophosphamide followed by paclitaxel or docetaxel. In some embodiments, the combination therapy is FEC:→T: 5-FU, epirubicin, and cyclophosphamide followed by docetaxel or paclitaxel. In some embodiments, the combination therapy is TC: docetaxel and cyclophosphamide. In some embodiments, the combination therapy is TCH: docetaxel, carboplatin, and trastuzumab for HER2/neu positive tumors. In some embodiments, the combination therapy is CMF: cyclophosphamide, methotrexate, and 5-fluorouracil. In some embodiments, the combination therapy is A→CMF: doxorubicin, followed by CMF. In some embodiments, the combination therapy is EC: epirubicin and cyclophosphamide. In some embodiments, the combination therapy is AC: doxorubicin and cyclophosphamide.

The compositions disclosed herein are formulated in any form suitable for delivery through the nipple of an individual. In some embodiments, the compositions are formulated in a hydroalcoholic gel, a hydroalcoholic solution, a patch, a cream, an emulsion, a lotion, an ointment, a powder, a paste, or an oil. In some embodiments, the composition is a hydroalcoholic gel or a hydroalcoholic solution. In some embodiments, the composition is an emulsion.

In some embodiments, the composition is emulsion in which therapeutics which are poorly soluble in water are dissolved in the oil. In some embodiments, the emulsion is an oil-in-alcohol emulsion, an alcohol-in-oil emulsion, an oil-in alcohol emulsion, an oil/alcohol/alcohol emulsion, oil-in-water emulsion, a water-in-oil emulsion, or a water-in-oil-in-water emulsion. In some preferred embodiments, the emulsion is an oil-in-water emulsion. In other preferred embodiments, the emulsion is an oil-in-alcohol emulsion or an oil/alcohol/water emulsion.

In some embodiments, the oil-in-water emulsion comprises an oil that is compatible for treatment of breast conditions. Suitable oils to use with the oil-in-water emulsion include, but are not limited to, soybean oil, medium-chain triglycerides, olive oil, and fish oils. Ins some embodiments, the oil-in-water emulsion is selected from Intralipid®, Liposyn® III, Ivelip®, Lipovenoes®, Lipovenoes® 10% PLR, Intralipos® 10%, Lipofundin-N®, Soyacal, Intrafat, Structolipid® 20%, Lipofundin® MCT/LCT, Lipovenoes® MCT, ClinOleic® 20%, Lipoplus®, SMOFlipid®, and Omegaven®.

### Diagnostic Agents

Radiocontrast Agents

In some embodiments, the diagnostic agent is a radiocontrast agent. As used herein, “radiocontrast agent” means any contrast agent which enables visualization of internal breast structures, e.g., breast ducts, via X-ray based imaging techniques such as computed tomography (CT) and radiography.

In some embodiments, the radiocontrast agent is an iodine compound. In some embodiments, the iodine compound is ionic. In some embodiments, the iodine compound is nonionic. In some embodiments, the contrast agent is acetrizoic acid, adipiodone (iodipamide), calcium iopodate, diatrizoate, diatrizoic acid (amidotrizoic acid; 3,5-diacetamido-2,4,6-triiodobenzoic acid; Hypaque; Gastrografin; Urografin), diodone, iobenzamic acid, iobitridol (Xenetix 300), iocarmic acid, iocetamic acid, iodixanol (Visipaque), iofendylate, ioglicic acid, ioglycamic acid, iohexol (Omnipaque), iomeprol, iopamidol (lopamiro, Isovue, lopamiron, and Niopam), iopanoic acid, iopentol, iopodate sodium (Oragrafin or Gastrografin), iopromide (Ultravist), iopydol, iotalamic acid, iotrolan (lsovist), iotroxic acid, ioversol, ioxaglic acid (Hexabrix), ioxilan (Oxilan), ioxitalamic acid (Telebrix), lipiodol (ethiodized oil; Ethiodol), methiodal, metrizamide, metrizoic acid, propyliodone (Dionosil), sodium iodamide, tyropanoic acid (Bilopaque, Lumopaque, Tyropaque, Bilopac), or any combinations thereof.

MRI Contrast Agents

In some embodiments, the diagnostic agent is a MRI contrast agent. As used herein, “MRI contrast agent” means any contrast agent which enables visualization of internal breast structures, e.g., breast ducts, via magnetic resonance imaging (MRI).

In some embodiments, the MRI contrast agent is a gadolinium (III) containing agent. In some embodiments, the MRI contrast agent is gadobenate (MultiHance), gadobutrol (Gadovist), gadodiamide (Omniscan), gadofosveset (Ablavar, formerly Vasovist), gadopentetate (Magnevist, Magnegita, Gado-MRT ratiopharm), gadoterate (Dotarem), gadoteridol (ProHance), gadoversetamide (OptiMARK), gadoxetate (Primovist, Eovist), or any combinations thereof.

In some embodiments, the MRI contrast agent is a gadolinium chelate. In some embodiments, the MRI contrast agent is diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), or combinations thereof.

In some embodiments, the MRI contrast agent is an iron oxide containing agent. In some embodiments, the MRI contrast agent is superparamagnetic iron oxide or ultrasmall superparamagnetic iron oxide. In some embodiments, the MRI contrast agent is ferucarbotran (Resovist), feruglose (Clariscan), ferumoxides injectable solution (Feridex LV.), ferumoxsil (Lumirem), ferumoxtran (Combidex, Sinerem), or any combinations thereof.

In some embodiments, the MRI contrast agent is superparamagnetic iron platinum.

In some embodiments, the MRI contrast agent is paramagnetic manganese.

Ultrasound Contrast Agents

In some embodiments, the diagnostic agent is an ultrasound contrast agent. As used herein, “ultrasound contrast agent” means any contrast agent which enables visualization of internal breast structures, e.g., breast ducts, via ultrasound. In some embodiments, the ultrasound contrast agent is a microbubble. In some embodiments, the ultrasound contrast agent perflexane lipid microspheres (Imagent, Imavist), perflutren lipid microspheres (Definity), galactose microparticles (Levovist), perflutren protein-type A microspheres (Optison), or any combinations thereof. In some embodiments, the ultrasound contrast agent is conjugated to a targeting moiety.

Radio Nuclides

In some embodiments, the diagnostic agent is a nuclear probe. In some embodiments, the diagnostic agent is a SPECT or PET radionuclide probe. In some embodiments, the radionuclide probe is selected from: a technetium chelate, a copper chelate, a radioactive fluorine, a radioactive iodine, and an indiuim chelate.

In some embodiments, the diagnostic agent is HYNIC, DTPA, and DOT A. In some embodiments, the diagnostic agent is 211At, 1311, 12SI, 90Y, 186Re, 188Re, 1S3Sm, 212Bi, 32P, 64Cu, a radioactive isotope of Lu, or any combinations thereof.

### Additional Components

In some embodiments, the composition comprises a dissolved gas. In some embodiments, the gas a high solubility in a cold liquid (e.g., between about 0° C. and 5° C.) and a low solubility in a liquid at room temperature. In some embodiments, the gas is carbon dioxide, oxygen, nitrogen, or any combinations thereof. In some embodiments, the gas is carbon dioxide. In some embodiments, the gas is oxygen. In some embodiments, the gas is nitrogen.

In some embodiments, the composition is refrigerated so that the dissolved gas stays in solution. In some embodiments, the composition is stored between 0° C. and 20° C. In some embodiments, the composition is stored between 0° C. and 15° C. In some embodiments, the composition is stored between 0° C. and 10° C. In some embodiments, the composition is stored between 0° C. and 5° C. In some embodiments, the composition is stored between 0° C. and 4° C. In some embodiments, the composition is stored between 0° C. and 2° C. In some embodiments, the composition is stored between 0° C. and 1.6° C.

### Kits

The present invention also relates to kits for delivering a composition to a breast duct of an individual in need thereof comprising devices and/or compositions disclosed herein. In some embodiments, the kit comprises a device for delivering a composition to a breast duct of the individual; a composition; and instructions for use of the device and the composition. In some embodiments, the devices comprise a treatment chamber comprising a composition. In some preferred embodiments, the composition further comprises at least one therapeutic agent. In some preferred embodiments, the composition further comprises a plurality of therapeutic agents. In some more preferred embodiments, the composition comprises endoxifen or a pharmaceutically acceptable salt thereof. In some more preferred embodiments, the composition comprises inhibitors of kynurenine pathway, or a combination thereof, or a pharmaceutically acceptable salt thereof. In some more preferred embodiments, the composition comprises endoxifen or a pharmaceutically acceptable salt thereof. In some more preferred embodiments, the composition comprises inhibitors of IDO1, IDO2, TDO, or a combination thereof, or a pharmaceutically acceptable salt thereof. In a more preferred embodiment, the kit comprises a device for delivering a composition to a breast duct of an individual in need thereof comprising a treatment chamber comprising a composition disclosed herein.

In some preferred embodiments, a kit comprises a device for delivering a composition to a breast duct of an individual in need thereof comprising a treatment chamber comprising a composition disclosed herein. In some preferred embodiments, a kit comprises: (a) a device for delivering a composition to a breast duct of an individual in need thereof comprising a treatment chamber comprising a composition comprising endoxifen or a pharmaceutically acceptable salt thereof; and instructions for use of the device. In other preferred embodiments, a kit comprises: a device for delivering a composition to a breast duct of an individual in need thereof comprising a treatment chamber comprising a composition comprising inhibitors of kynurenine pathway or a pharmaceutically acceptable salt thereof; and instructions for use of the device. In still other preferred embodiments, a kit comprises: a device for delivering a composition to a breast duct of an individual in need thereof comprising a treatment chamber comprising a composition comprising inhibitors of IDO1, IDO2, TDO, or a combination thereof, or pharmaceutically acceptable salts thereof; and instructions for use of the device.

In some embodiments, the invention provides a dose, a unit dose, or multiple dose of the pharmaceutical dose package. In some embodiments, the packaging reflects a dosing regimen or schedule of application, such as twice daily, daily, weekly, twice weekly, biweekly, monthly, quarterly, 6 monthly, or yearly application.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

### EXAMPLES

### Example 1. Permeation of Endoxifen by Skin Models

This example describes an in vitro skin model for the study of endoxifen permeation.

Porcine Skin Model

Strips of porcine sow breasts are obtained post mortem from a local abattoir and transported to laboratory in iced HEPES modified Hanks buffered balanced salt solution (HBBS). The freshly excised strips of the porcine mammary papillae are washed in tepid water and the mammary papilla, surrounded by 2 cm×2 cm of abdominal skin, are excised by blunt dissection. Cutaneous fatty tissue is also removed by blunt dissection and the pieces are maintained in HHBS until prepared in diffusion cells.

In vitro trans-mammary papilla delivery is tested using the Franz diffusion cell system consisting of 2 separate chambers, a donor chamber and a receptor chamber. The porcine breast samples are mounted between the cell compartments, the flanges of which are smeared with high vacuum silicon grease with the mammary papilla located in the center and facing upwards as described by Lee et al. (International J. Pharmaceutics. 2010, 387, 161-166). The 2 chambers are held together with a clamped to minimize leakage. The receptor chamber has a volume of about 4.3 mL and is filled with receptor fluid via a sampling arm. Micro-stir bars are added and the complete diffusion cells are placed on a submersible magnetic stirrer base set up in a water bath at 37° C. The breast is mounted in the horizontal position, and the donor is sealed with a greased microscope slide and the cell rotated 90° and supported as necessary. After 30 minutes, 500 μL of the composition, described in Table 1 below, is applied to the surface of the skin by means of a pipette, or a spatula where the hydroalcoholic gel is used. The donor compartment is occluded with laboratory film (n=4).

TABLE 1

| Ingredient | Quantity per 100 g emulsion |
|---|---|
| Endoxifen (E/Z isomer mix) | 0.5 g and 1 g |
| Isopropyl myristate, US USP | 1 g |
| Ethanol | 30% |
| Surfactant - Cremaphor | 1% |
| Fish Oil | q.s. 100 g |

After 6 hours, the diffusion cells are dismantled and the breast tissue is recovered. Excess dose and grease are wiped away and the diffused cells are excised and centrifuged at 10,000×g to remove excess solution and gel, cut into approximately 1 mm×1 mm×1 mm cubes with a scalpel and placed into a 5 mL centrifuge tube. 2 mL of methanol is added and the tube is vortex mixed for 30 seconds before being placed on a rotating blood cell mixer for 30 minutes. The tubes are centrifuged at 10,000×g and the supernatant is decanted into 10 mL glass bottles. Additional aliquots of methanol are added and the extraction process is repeated twice more before the pooled supernatants are reduced in a vacuum oven set at 50° C. The residues are then reconstituted with a 1 mL of HPLC mobile phase.

The samples are analyzed by reverse-phase liquid chromatography. Analytes are separated, and statistical analyses is performed on the results. Drug delivery into the papilla oriented vertically is compared with that delivered into the papilla oriented laterally by means of Wilcoxon match-pairs signed-ranks test, and the combined formulation of the samples is compared via a Kruskal-Wallis non-parametric ANAOVA test (Instat 3, GraphPad software, CA, USA). In some samples, nipple tissue is separately assessed and compared with the areolae. Confidence intervals are set at 95% and p<0.05 is deemed as statistically significant.

### Example 2. Transpapillary Delivery of Endoxifen Gel

An individual diagnosed with hyperplasia in two breast ducts (one duct in her right breast and one duct in her left breast) is treated with endoxifen delivered by transpapillary method as follows. In the following non-limiting example, the individual is diagnosed with hyperplasia using nipple aspirate fluid (NAP). Alternatively, another diagnostic method can be used, such as mammography. Keratin plugs in the breast ducts are removed. Sufficient amount of NAP is collected from each breast of the subject. The sample from each breast is analyzed using cytology tests and determining the expression pattern of cancer biomarkers CK5, CK14, CK7, CK18, and p53 using antibodies directed against these biomarkers. The analysis reveals ductal hyperplastic disorder in two suspect ducts.

Each of the individual's nipples that contains the suspect duct is wiped with alcohol and is air dried prior to treatment. Next, the nipples are contacted with the treatment chamber of the device disclosed in U.S. Pat. No. 6,629,936, which is capable of forcing a composition into the breast duct under pressure. Another device capable of delivering a composition through the individual's nipple under positive pressure can also be used.

The treatment chamber of the device contains endoxifen gel. The endoxifen gel formulation is provided below in Table 2.

TABLE 2

| Ingredient | Quantity per 100 g of gel |
|---|---|
| Endoxifen (E/Z isomer mix) | 0.5 g and 1 g |
| Isopropyl palmitate, US USP | 1 g |
| HPMC | 1 g |
| Fish Oil | q.s. 100 g |

The endoxifen gel described above is spiked with the MRI contrasting agent Gadolimium contrast agent (5 mM). Another MRI contrasting agent can also be used.

The device delivers 1 mL of the endoxifen gel into the breast ducts through the nipple under positive pressure. Following treatment, the breast and the nipple are wiped clean. Functional T1 magnetic resonance imaging (MRI) of the breast is performed to visualize the ducts and evaluate the localized delivery of the endoxifen gel close to the affected sites.

Localized transpapillary delivery of the drug to the breast ducts closer to the site of the breast hyperplasia presents low systemic exposure of the subject to the drug. The individual's blood is drawn to measure the levels of endoxifen in the blood plasma.

References are made in detail to certain embodiments of the invention, examples of which are illustrated herein. It is intended that any and all parts of the disclosure can be read in combination with any other part of the disclosure, unless otherwise apparent from the text. While the invention is described in conjunction with the enumerated embodiments, it will be understood that they are not intended to limit the invention to those embodiments. It is specifically intended to cover all alternatives, modifications, and equivalents which can be included within the scope of the present invention as defined by the claims. At various places in the present specification, substituents of compounds of the invention can be disclosed in groups. It is specifically intended that the invention include each and every individual sub-combination of the members of such groups.

It is further appreciated that certain features of the invention, which are, for clarity, described in the context of separate embodiments, can also be provided in combination in a single embodiment. Conversely, various features of the invention which are, for brevity, described in the context of a single embodiment can also be provided separately or in any suitable sub-combination.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compounds and compositions disclosed herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A method of delivering a composition comprising endoxifen or a pharmaceutically acceptable salt thereof to a breast duct of an individual in need thereof, comprising:
(a) contacting the composition contained within a treatment chamber of a device with a nipple of a breast; and
(b) applying positive pressure on the composition.

2. The method of claim 1, wherein the composition is forced into 1 to 5 breast ducts, preferably, into 4 to 8 breast ducts, and more preferably, into 7 to 11 breast ducts.

3. The method of claim 1, wherein the individual has a breast disorder.

4. The method of claim 3, wherein the breast disorder is proliferative breast disease, breast cancer, or increased breast density.

5. The method of claim 3, wherein the proliferative breast disease is atypical ductal hyperplasia or atypical lobular hyperplasia.

6. The method of claim 4, wherein the breast is cancer ductal carcinoma in situ, lobular carcinoma in situ, invasive (or infiltrating) ductal carcinoma, invasive (or infiltrating) lobular carcinoma, or inflammatory breast cancer.

7. The method of claim 4, wherein the breast cancer is ER+ breast cancer, HER2+ breast cancer, or triple-negative breast cancer.

8. The method of claim 4, wherein the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma.

9. The method of claim 1, wherein the individual is tamoxifen resistant.

10. The method of claim 1, wherein the individual has been predicted to have a (i) moderate to high risk of cancer relapse or (ii) low to moderate rate of disease-free survival.

11. The method of claim 10, wherein the moderate or high risk of cancer relapse is due to reduced expression or reduced function of a member of cytochrome P450 family.

12. The method of claim 11, wherein the member of cytochrome P450 family is CYP2D6, CYP2B6, CYP2C9, CYP2C19, CYP3A4, or CYP3A5.

13. The method of claim 1, wherein the individual has immune suppression.

14. The method of claim 1, wherein the individual has a high risk of tumor escape.

15. The method of claim 1, wherein the individual has increased activity or expression of IDO1, IDO2, TDO, or a combination thereof.

16. The method of claim 1, wherein the endoxifen is an E-isomer, a Z-isomer or a mixture thereof.

17. The method of claim 1, wherein the composition comprises endoxifen or a pharmaceutically acceptable salt thereof at a concentration ranging from 0.01% to 15% by weight of the composition.

18. The method of claim 1, wherein the composition comprising endoxifen or a pharmaceutically acceptable salt thereof is formulated in a hydroalcoholic gel, a hydroalcoholic solution, a patch, a cream, an emulsion, a lotion, an ointment, a powder, a paste, or an oil.

19. The method of claim 1, wherein the composition further comprises at least one therapeutic agent.

20. The method of claim 1, wherein the composition further comprises a plurality of therapeutic agents.

21. The method of claim 19, wherein the therapeutic agent is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anticancer antibodies, immunotherapy agents, anthracyclins, platinums, vinca alkoids, camptothecins, taxanes, hormones, 1-alpha-hydroxylase inhibitors, 24-hydroxylase inhibitors, or a combination thereof.

22. The method of claim 19, wherein the composition further comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, 4-hydroxytamoxifen, N-desmethyltamoxifen, endocoxifen, lasofoxifene, raloxifene, benzothiophene, bazedofoxifene, arzoxifene, miproxifene, levormeloxifene, droloxifene, clomifene, idoxifene, EM652, ERA-92, toremifene, trastuzumab, vinorelbine, butyric acid, epirubicin, doxorubicin, and combinations thereof.

23. The method of claim 19, wherein the therapeutic agent is a tryptophan metabolism inhibitor, a kynurenine depletor, an inhibitor of IDO1, IDO2, TDO, or a combination thereof.

24. The method of claim 23, wherein the therapeutic agent is an inhibitor of IDO1, IDO2, TDO, or a combination thereof.

25. The method of claim 19, wherein the therapeutic agent is a PD-L1 inhibitor, a PD-1 inhibitor, a CTLA-4 inhibitor, or a combination thereof.

26. The method of claim 1, wherein the composition further comprises at least one omega-3 fatty acid, at least one vitamin D compound or a pharmaceutically acceptable salt thereof, or a combination thereof.

27. The method of claim 1, wherein the composition comprises (i) endoxifen or a pharmaceutically acceptable salt thereof; (ii) at least one an omega-3 fatty acid; and (iii) at least one vitamin D compound or a pharmaceutically acceptable salt thereof.

28. The method of claim 27, wherein the composition comprises the omega-3 fatty acid at a concentration ranging from 10% to 90% by weight of the composition.

29. The method of claim 26, wherein the omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof.

30. The method of claim 26, wherein the omega-3 fatty acid is a triglyceride or a phospholipid.

31. The method of claim 1, wherein the composition further comprises a mixture of EPA and DHA.

32. The method of claim 26, the vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4, 1,25 dihydroxy-19-nor-vitamin D2, 1-alphahydroxyvitamin D3, vitamin D analogs, and a combination thereof.

33. The method of claim 26, wherein the vitamin D compound is cholecalciferol.

34. The method of claim 26, wherein the vitamin D compound has an activity ranging from 10 IU to 6000 IU, preferably from 100 IU to 4000 IU, more preferably from 200 IU to 2000 IU, even more preferably from 400 IU to 1000 IU, and still more preferably from 10 IU to 200 IU.

35. The method of claim 1, wherein the composition has a low viscosity.

36. The method of claim 1, wherein the composition has a viscosity of less than 10 cp, 5 cp, or 1 cp at 25° C.

37. The method of claim 1, wherein the composition further comprises dissolved carbon dioxide.

38. The method of claim 37, wherein the positive pressure is applied to the composition by the escape of the carbon dioxide from the composition as the temperature of the composition increases.

39. The method of claim 1, wherein the composition is contacted with the nipple of a breast on the 2^{nd} week of the individual's menstrual cycle.

40. The method of claim 1, wherein the composition is contacted with the nipple of a breast for at least 6 hours, 8 hours, 10 hours, 12 hours, 18 hours, or 24 hours.

41. The method of claim 1, further comprising applying a topical anesthetic to the nipple before the composition is contacted with the nipple.

42. The method of claim 1, further comprising cleaning the nipple before the composition is contacted with the nipple.

43. The method of claim 1, wherein the device further comprises: a first opening sized to circumscribe a nipple, which opening is operatively connected to the treatment chamber.

44. The method of claim 1, wherein the device further comprises: a second opening operatively connected to the treatment chamber through which through which the composition is instilled into the treatment chamber.

45. The method of claim 1, wherein the device further comprises a third opening operatively connected to the treatment chamber through which positive pressure is applied to the composition.

46. The method of claim 1, further comprising adhering the device to the nipple.

47. The method of claim 1, wherein the device further comprises an adhesive which adheres the device to the breast.

48. The method according to claim 47, wherein the adhesive is a silicone-based skin adhesive.

49. The method of claim 1, further comprising applying a cover over the nipple after removing the device.

50. The method of claim 49, wherein the cover is waterproof and/or airtight and/or opaque.

51. The method of claim 49, wherein the cover comprises a liquid bandage.

52. The method of claim 49, wherein the cover comprises a patch.

53. The method of claim 49, wherein the cover comprises a film.

54. The method of claim 49, wherein the cover comprises an occlusive agent.

55. The method of claim 49, wherein the cover comprises an anti-inflammatory agent, an antioxidant, or an antiseptic.

56. A method of treating a breast disorder, comprising:
(a) contacting a treatment chamber of a device comprising a composition comprising endoxifen or a pharmaceutically acceptable salt thereof with a nipple of a breast of an individual; and
(b) applying positive pressure on the composition.

57. The method of claim 56, wherein the composition is forced into 1 to 5 breast ducts, preferably, into 4 to 8 breast ducts, and more preferably, into 7 to 11 breast.

58. The method of claim 56, wherein the breast disorder is proliferative breast disease, breast cancer or increased breast density.

59. The method of claim 56, wherein the proliferative breast disease is atypical ductal hyperplasia or atypical lobular hyperplasia.

60. The method of claim 58, wherein the breast cancer is ductal carcinoma in situ, lobular carcinoma in situ, invasive (or infiltrating) ductal carcinoma, invasive (or infiltrating) lobular carcinoma, or inflammatory breast cancer.

61. The method of claim 58, wherein the breast cancer is ER+ breast cancer, HER2+ breast cancer, or triple-negative breast cancer.

62. The method of claim 58, wherein the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcmoma.

63. The method of claim 56, wherein the individual is tamoxifen resistant.

64. The method of claim 56, wherein the individual has been predicted to have a (i) moderate to high risk of cancer relapse or (ii) low to moderate rate of disease-free survival.

65. The method of claim 66, wherein the moderate or high risk of cancer relapse is due to reduced expression or reduced function of a member of cytochrome P450 family.

66. The method of claim 65, wherein the member of cytochrome P450 family is CYP2D6, CYP2B6, CYP2C9, CYP2C19, CYP3A4, or CYP3A5.

67. The method of claim 56, wherein the individual has immune suppression.

68. The method of claim 56, wherein the individual has a high risk of tumor escape.

69. The method of claim 56, wherein the individual has increased activity or expression of IDO1, IDO2, TDO, or a combination thereof.

70. The method of claim 56, wherein the endoxifen is an E-isomer, a Z-isomer or a mixture thereof.

71. The method of claim 56, wherein the composition comprises endoxifen or a pharmaceutically acceptable salt thereof at a concentration ranging from 0.01% to 15% by weight of the composition.

72. The method of claim 56, wherein the composition comprising endoxifen is formulated in a hydroalcoholic gel, a hydroalcoholic solution, a patch, a cream, an emulsion, a lotion, an ointment, a powder, a paste, or an oil.

73. The method of claim 56, wherein the composition further comprises at least one therapeutic agent.

74. The method of claim 56, wherein the composition further comprises a plurality of therapeutic agents.

75. The method of claim 73, wherein the therapeutic agent is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anticancer antibodies, immunotherapy agents, anthracyclins, platinums, vinca alkoids, camptothecins, taxanes, hormones, 1-alpha-hydroxylase inhibitors, 24-hydroxylase inhibitors, or a combination thereof.

76. The method of claim 73, wherein the composition further comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, 4-hydroxytamoxifen, N-desmethyltamoxifen, endocoxifen, lasofoxifene, raloxifene, benzothiophene, bazedofoxifene, arzoxifene, miproxifene, levormeloxifene, droloxifene, clomifene, idoxifene, EM652, ERA-92, toremifene, trastuzumab, vinorelbine, butyric acid, epirubicin, doxorubicin, and combinations thereof.

77. The method of claim 73, wherein the therapeutic agent is a tryptophan metabolism inhibitor, a kynurenine depletor, an inhibitor of IDO1, IDO2, TDO, or a combination thereof.

78. The method of claim 77, wherein the therapeutic agent is an inhibitor of IDO1, IDO2, TDO, or a combination thereof.

79. The method of claim 73, wherein the therapeutic agent is a PD-L1 inhibitor, a PD-1 inhibitor, a CTLA-4 inhibitor, or a combination thereof.

80. The method of claim 56, wherein the composition further comprises at least one omega-3 fatty acid, at least one vitamin D compound or a pharmaceutically acceptable salt thereof, or a combination thereof.

81. The method of claim 56, wherein the composition comprises (i) endoxifen or a pharmaceutically acceptable salt thereof; (ii) at least one an omega-3 fatty acid; and (iii) at least one vitamin D compound or a pharmaceutically acceptable salt thereof.

82. The method of claim 80, wherein the composition comprises the omega-3 fatty acid at a concentration ranging from 10% to 90% by weight of the composition.

83. The method of claim 80, wherein the omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof.

84. The method of claim 80, wherein the omega-3 fatty acid is a triglyceride or a phospholipid.

85. The method of claim 56, wherein the composition further comprises a mixture of EPA and DHA.

86. The method of claim 80, the vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4, 1,25 dihydroxy-19-nor-vitamin D2, 1-alphahydroxyvitamin D3, vitamin D analogs, and a combination thereof.

87. The method of claim 80, wherein the vitamin D compound is cholecalciferol.

88. The method of claim 80, wherein the vitamin D compound has an activity ranging from 10 IU to 6000 IU, preferably from 100 IU to 4000 IU, more preferably from 200 IU to 2000 IU, even more preferably from 400 IU to 1000 IU, and still more preferably from 10 IU to 200 IU.

89. The method of claim 56, wherein the composition has a low viscosity.

90. The method of claim 56, wherein the composition has a viscosity of less than 10 cp, 5 cp, or 1 cp at 25° C.

91. The method of claim 56, wherein the composition further comprises dissolved carbon dioxide.

92. The method of claim 91, wherein the positive pressure is applied to the composition by the escape of the carbon dioxide from the composition as the temperature of the composition increases.

93. The method of claim 56, wherein the composition is contacted with the nipple of a breast on the 2^{nd} week of the individual's menstrual cycle.

94. The method of claim 56, wherein the composition is contacted with the nipple of a breast for at least 6 hours, 8 hours, 10 hours, 12 hours, 18 hours, or 24 hours.

95. The method of claim 56, further comprising applying a topical anesthetic to the nipple before the composition is contacted with the nipple.

96. The method of claim 56, further comprising cleaning the nipple before the composition is contacted with the nipple.

97. The method of claim 56, wherein the device further comprises:
(a) a first opening sized to circumscribe a nipple, which opening is operatively connected to the treatment chamber; and
(b) a second opening operatively connected to the treatment chamber through which positive pressure is applied to the composition comprising endoxifen or a pharmaceutically acceptable salt thereof.

98. The method of claim 97, wherein the device further comprises a third opening through which the composition comprising at least one therapeutic agent is instilled into the treatment chamber.

99. The method of claim 56, further comprising adhering the device to the nipple.

100. The method of claim 56, wherein the device further comprises an adhesive which adheres the device to the breast.

101. The method of claim 56, further comprising applying a cover over the nipple after removing the device.

102. The method of claim 101, wherein the cover is waterproof and/or airtight and/or opaque.

103. The method of claim 101, wherein the cover comprises a liquid bandage.

104. The method of claim 101, wherein the cover comprises a patch.

105. The method of claim 101, wherein the cover comprises a film.

106. The method of claim 101, wherein the cover comprises an occlusive agent.

107. The method of claim 101, wherein the cover comprises an anti-inflammatory agent, an anti-oxidant, or an antiseptic.

108. A composition for use in the treatment of a breast disorder in an individual, comprising (a) endoxifen or a pharmaceutically acceptable salt thereof, and (b) a dissolved gas.

109. The composition of claim 108, wherein the dissolved gas is carbon dioxide.

110. The composition of claim 108, wherein the individual has a breast disorder.

111. The composition of claim 110, wherein the breast disorder is a proliferative breast disease, a breast cancer, or increased breast density.

112. The composition of claim 111, wherein the proliferative breast disease is atypical ductal hyperplasia or atypical lobular hyperplasia.

113. The composition of claim 111, wherein the breast cancer is ductal carcinoma in situ, lobular carcinoma in situ, invasive (or infiltrating) ductal carcinoma, invasive (or infiltrating) lobular carcinoma, or inflammatory breast cancer.

114. The composition of claim 111, wherein the breast cancer is ER+ breast cancer, HER2+ breast cancer, or triple-negative breast cancer.

115. The composition of claim 111, wherein the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma.

116. The composition of claim 108, wherein the individual is tamoxifen resistant.

117. The composition of claim 108, wherein the individual has been predicted to have a (i) moderate to high risk of cancer relapse or (ii) low to moderate rate of disease-free survival.

118. The composition of claim 117, wherein the moderate or high risk of cancer relapse or low to moderate rate of disease-free survival is due to a:
(a) reduced expression or reduced function of a member of cytochrome P450 family; or
(b) increased expression or an increased activity of IDO1, IDO2, TDO, or a combination thereof.

119. The composition of claim 118, wherein the member of cytochrome P450 family is CYP2D6, CYP2B6, CYP2C9, CYP2C19, CYP3A4, or CYP3A5.

120. The composition of claim 108, wherein the individual has immune suppression.

121. The composition of claim 108, wherein the individual has a high risk of tumor escape.

122. The composition of claim 108, wherein the individual has increased activity or expression of IDO1, IDO2, TDO, or a combination thereof.

123. The composition of claim 108, wherein the endoxifen is an E-isomer, a Z-isomer or a mixture thereof.

124. The composition of claim 108, wherein the composition comprises endoxifen or a pharmaceutically acceptable salt thereof at a concentration ranging from 0.01% to 15% by weight of the composition.

125. The composition of claim 108, wherein the composition comprising endoxifen is formulated in a hydroalcoholic gel, a hydroalcoholic solution, a patch, a cream, an emulsion, a lotion, an ointment, a powder, a paste, or an oil.

126. The composition of claim 108, wherein the composition further comprises at least one therapeutic agent.

127. The composition of claim 108, wherein the composition further comprises a plurality of therapeutic agents.

128. The composition of claim 126, wherein the therapeutic agent is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anticancer antibodies, immunotherapy agents, anthracyclins, platinums, vinca alkoids, camptothecins, taxanes, hormones, 1-alpha-hydroxylase inhibitors, 24-hydroxylase inhibitors, or a combination thereof.

129. The composition of claim 126, wherein the composition further comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, 4-hydroxytamoxifen, N-desmethyltamoxifen, endocoxifen, lasofoxifene, raloxifene, benzothiophene, bazedofoxifene, arzoxifene, miproxifene, levormeloxifene, droloxifene, clomifene, idoxifene, EM652, ERA-92, toremifene, trastuzumab, vinorelbine, butyric acid, epirubicin, doxorubicin, and combinations thereof.

130. The composition of claim 126, wherein the therapeutic agent is a tryptophan metabolism inhibitor, a kynurenine depletor, an inhibitor of IDO1, IDO2, TDO, or a combination thereof.

131. The composition of claim 126, wherein the therapeutic agent is an inhibitor of IDO1, IDO2, TDO, or a combination thereof.

132. The composition of claim 126, wherein the therapeutic agent is a PD-L1 inhibitor, a PD-1 inhibitor, a CTLA-4 inhibitor, or a combination thereof.

133. The composition of claim 108, wherein the composition further comprises at least one omega-3 fatty acid, at least one vitamin D compound or a pharmaceutically acceptable salt thereof, or a combination thereof.

134. The composition of claim 108, wherein the composition comprises (i) endoxifen or a pharmaceutically acceptable salt thereof; (ii) at least one an omega-3 fatty acid; and (iii) at least one vitamin D compound or a pharmaceutically acceptable salt thereof.

135. The composition of claim 133, wherein the composition comprises the omega-3 fatty acid at a concentration ranging from 10% to 90% by weight of the composition.

136. The composition of claim 133, wherein the omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof.

137. The composition of claim 133, wherein the composition comprises a mixture of EPA and DHA.

138. The composition of claim 133, wherein the omega-3 fatty acid is a triglyceride or a phospholipid.

139. The composition of claim 133, the vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4, 1,25 dihydroxy-19-nor-vitamin D2, 1-alphahydroxyvitamin D3, vitamin D analogs, and a combination thereof.

140. The composition of claim 133, wherein the vitamin D compound is cholecalciferol.

141. The composition of claim 133, wherein the vitamin D compound has an activity ranging from 10 IU to 6000 IU, preferably from 100 IU to 4000 IU, more preferably from 200 IU to 2000 IU, even more preferably from 400 IU to 1000 IU, and still more preferably from 10 IU to 200 IU.

142. The composition of claim 108, wherein the composition has a low viscosity.

143. The composition of claim 107, wherein the composition has a viscosity of less than 10 cp, 5 cp, or 1 cp at 25° C.

144. The composition of claim 108, wherein the composition further comprises dissolved carbon dioxide.

145. The composition of claim 144, wherein the positive pressure is applied to the composition by the escape of the carbon dioxide from the composition as the temperature of the composition increases.

146. The composition of claim 108, wherein the composition is contacted with the nipple of a breast on the 2^{nd} week of the individual's menstrual cycle.

147. The composition of claim 108, wherein the composition is contacted with the nipple of a breast for at least 6 hours, 8 hours, 10 hours, 12 hours, 18 hours, or 24 hours.

148. A device for delivering a composition to a breast duct of an individual in need thereof, comprising:
(a) a treatment chamber;
(b) a first opening sized to circumscribe a nipple, which opening is operatively connected to the treatment chamber; and
(c) a composition comprising endoxifen or a pharmaceutically acceptable salt thereof.

149. The device of claim 148, wherein the composition comprising the endoxifen or a pharmaceutically acceptable salt thereof is contained within the treatment chamber.

150. The device of claim 148, further comprising a second opening operatively connected to the treatment chamber through which through which the composition is instilled into the treatment chamber.

151. The device of claim 150, further comprising a third opening operatively connected to the treatment chamber through which positive pressure is applied to the composition.

152. The device of claim 148, wherein the endoxifen is an E-isomer, a Z-isomer or a mixture thereof.

153. The device of claim 148, wherein the composition comprises endoxifen or a pharmaceutically acceptable salt thereof at a concentration ranging from 0.01% to 15% by weight of the composition.

154. The device of claim 148, wherein the composition comprising endoxifen is formulated in a hydroalcoholic gel, a hydroalcoholic solution, a patch, a cream, an emulsion, a lotion, an ointment, a powder, a paste, or an oil.

155. The device of claim 148, wherein the composition further comprises at least one therapeutic agent.

156. The device of claim 148, wherein the composition comprises a plurality of therapeutic agents.

157. The device of claim 155, wherein the therapeutic agent is selected from the group consisting of alkylating agents, anti-neoplastics, anti-mimetics, anti-metabolites, anti-tumor antibiotics, topoisomerase inhibitors, mitotic inhibitors, corticosteroids, differentiating agent, anticancer antibodies, immunotherapy agents, anthracyclins, platinums, vinca alkoids, camptothecins, taxanes, hormones, 1-alpha-hydroxylase inhibitors, 24-hydroxylase inhibitors, or a combination thereof.

158. The device of claim 155, wherein the composition further comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, 4-hydroxytamoxifen, N-desmethyltamoxifen, endocoxifen, lasofoxifene, raloxifene, benzothiophene, bazedofoxifene, arzoxifene, miproxifene, levormeloxifene, droloxifene, clomifene, idoxifene, EM652, ERA-92, toremifene, trastuzumab, vinorelbine, butyric acid, epirubicin, doxorubicin, and combinations thereof.

159. The device of claim 155, wherein the therapeutic agent is a tryptophan metabolism inhibitor, a kynurenine depletor, an inhibitor of IDO1, IDO2, TDO, or a combination thereof.

160. The device of claim 155, wherein the therapeutic agent is an inhibitor of IDO1, IDO2, TDO, or a combination thereof.

161. The device of claim 155, wherein the therapeutic agent is a PD-L1 inhibitor, a PD-1 inhibitor, a CTLA-4 inhibitor, or a combination thereof.

162. The device of claim 155, wherein the composition further comprises at least one omega-3 fatty acid, at least one vitamin D compound or a pharmaceutically acceptable salt thereof, or a combination thereof.

163. The device of claim 155, wherein the composition further comprises (i) endoxifen or a pharmaceutically acceptable salt thereof; (ii) at least one an omega-3 fatty acid; and (iii) at least one vitamin D compound or a pharmaceutically acceptable salt thereof.

164. The device of claim 163, wherein the composition comprises the omega-3 fatty acid at a concentration ranging from 10% to 90% by weight of the composition.

165. The device of claim 161, wherein the omega-3 fatty acid is selected from a group consisting of an EPA, a DHA, an ALA, an HTA, a SDA, an ETE, an ETA, an EPA, an HPA, a DPA, a clupanodonic acid, a tetracosapentaenoic acid, a tetracosahexaenoic acid, nisinic acid, and a combination thereof.

166. The device of claim 162, wherein the composition comprises a mixture of EPA and DHA.

167. The device of claim 162, wherein the omega-3 fatty acid is a triglyceride or a phospholipid.

168. The device of claim 162, the vitamin D compound is selected from the group consisting of calciferol, cholecalciferol, ergocalciferol, vitamin D metabolites, 25 hydroxyvitamin D3, 25 hydroxyvitamin D2, 25(OH)D, 1,25(OH)(2)D, 25 hydroxyvitamin D4, 25 hydroxyvitamin D5, 25 hydroxyvitamin D7, 1-alpha-25 hydroxyvitamin D3, 1-alpha-25 hydroxyvitamin D2, 1-alpha-25 hydroxyvitamin D4, 1,25 dihydroxy-19-nor-vitamin D2, 1-alphahydroxyvitamin D3, vitamin D analogs, and a combination thereof.

169. The device of claim 162, wherein the vitamin D compound is cholecalciferol.

170. The device of claim 162, wherein the vitamin D compound has an activity ranging from 10 IU to 6000 IU, preferably from 100 IU to 4000 IU, more preferably from 200 IU to 2000 IU, even more preferably from 400 IU to 1000 IU, and still more preferably from 10 IU to 200 IU.

171. The device of claim 148, wherein the composition has a low viscosity.

172. The device of claim 148, wherein the composition has a viscosity of less than 10 cp, 5 cp, or 1 cp at 25° C.

173. The device of claim 148, wherein the composition further comprises dissolved carbon dioxide.

174. The device of claim 173, wherein the positive pressure is applied to the composition by the escape of the carbon dioxide from the composition as the temperature of the composition increases.

175. The device of claim 148, wherein the composition is contacted with the nipple of a breast on the 2^{nd} week of the individual's menstrual cycle.

176. The device of claim 148, wherein the composition is contacted with the nipple of a breast for at least 6 hours, 8 hours, 10 hours, 12 hours, 18 hours, or 24 hours.

177. The device of claim 148, further comprising applying a topical anesthetic to the nipple before the composition is contacted with the nipple.

178. The device of claim 148, further comprising cleaning the nipple before the composition is contacted with the nipple.

179. The device of claim 148, further comprising adhering the device to the nipple.

180. The device of claim 148, wherein the device further comprises an adhesive which adheres the device to the breast.

181. A kit for delivering a composition to a breast duct of an individual in need thereof, comprising: a device for delivering a composition to a breast duct of the individual; a composition comprising endoxifen or a pharmaceutically acceptable salt thereof; and instructions for use of the device and the composition.

182. The kit of claim 181, wherein the device further comprises a treatment chamber comprising the composition.

183. The kit of claim 181, wherein the composition further comprises at least one therapeutic agent.

184. The kit of claim 181, wherein the composition further comprises a plurality of therapeutic agents.
